(19) United States
(12) Patent Application Publication
(10) Pub. No.: US 2022/0246251 A1
(43) Pub. Date: Aug. 4, 2022

(54) COUPLED MATRIX-MATRIX AND COUPLED TENSOR-MATRIX COMPLETION METHODS FOR PREDICTING DRUG-TARGET INTERACTIONS

(71) Applicant: THE REGENTS OF THE UNIVERSITY OF MICHIGAN, Ann Arbor, MI (US)

(72) Inventors: Maryam Bagherian, Ann Arbor, MI (US); Kayvan Najarian, Northville, MI (US); Harm Derksen, Dexter, MI (US)

(21) Appl. No.: 17/204,836

(22) Filed: Mar. 17, 2021

Related U.S. Application Data
(60) Provisional application No. 62/991,471, filed on Mar. 18, 2020.

(51) Int. Cl. G16C 20/50

(52) U.S. Cl. CPC G16C 20/50

(57) ABSTRACT

Techniques for predicting drug and target interactions in incomplete matrices are provided for use in new drug discovery and drug repurposing. Matrix completion is achieved through matrix factorization that employs coupled matrix-matrix completion processes capable of completing a drug-target interaction matrix using coupled input matrices of each dataset. Matrix completion techniques also extend to using coupled tensors containing multiple slices of each dataset and using coupled tensor-matrix completion techniques for predicting drug and target interactions.

### CROSS-REFERENCE TO RELATED APPLICATION

Priority is claimed to U.S. Provisional Patent Application No. 62/991,471, filed Mar. 18, 2020, the entire disclosure of which is incorporated herein by reference.

### FIELD OF THE DISCLOSURE

The invention generally relates to method of predicting associations between multiple datasets and, in particular, between drug databases and target databases for predicting drug-target interactions.

### BACKGROUND

The background description provided herein is for the purpose of generally presenting the context of the disclosure. Work of the presently named inventor, to the extent it is described in this background section, as well as aspects of the description that may not otherwise qualify as prior art at the time of filing, are neither expressly nor impliedly admitted as prior art against the present disclosure.

Many drug treatments result from carefully scrutinized testing under regulated conditions, where researchers track patient demographic data, patient pathology data, drug responsiveness data, and the like. While traditionally, clinical testing is used to identify potential drug treatments and their predictive efficacy of various pathologies, increasingly researcher are resorting to statistical analyses in the hopes of identifying potential drug treatments and predicting their efficacy. In contrast to in vivo and in vitro testing, these statistically analyses are in silico and involve comparing massive datasets, such as comparing datasets of drug treatments against datasets of target genes or proteins, to identify treatment candidates for patient cohorts in which no previous clinical data is available, or in which the data that is available is of little statistical significance.

Recent statistical analysis techniques include the development of in silico drug-target interaction (DTI) prediction methods. That is, although in vitro experiments are the ultimate step in the drug discovery process, computational predictions are increasingly viewed as important to avoid expensive, laborious lab, and time consuming experiments early in the drug identification process. To this end, recent interest has turned to machine learning based and other prediction methods for early pharmacological DTI predictions. For example, machine learning techniques have been proposed to compensate for the lack of three-dimensional (3D) structures of drugs and of targets, in order to identify potential binding events that could hinder drug efficacy. The ability to model binding effects of a drug and target is particularly valuable in immunotherapies and with monoclonal antibody drugs.

Many DTI prediction methods incorporate drug-drug or target-target structural relationships using techniques called Similarity/Distance-Based Methods. The main disadvantages of such approaches are that they are sensitive to the fact that only a small percent of drugs have known interactions and typically drug-target interaction datasets comprise binary data, even though drug-target binding affinities are continuous in nature. Another family of solutions for DTI prediction methods are Network-Based Methods that utilize graph-based techniques to perform DTI prediction. Although some methods use three networks of protein-protein similarity, drug-drug similarity, and known drug-target interactions in a heterogeneous network, these methods tend to perform poorly in DTI discovery, to date. These deficiencies may be due to the fact that the properties of DTI networks are not favorable for such methods.

Another method for DTI prediction is a Feature-Based Method, which has recently been used in DTI prediction tasks. Feature-Based methods include machine learning techniques such as support vector machines (SVM), tree-based methods, and other kernel based methods used with 3D protein structures. Conceptually, any drug-target pair can be represented in terms of a feature vector, often with binary labels, and a machine learning method may be used to classify the pair-vectors into positive or negative interacting proteins prevents extracting the main features disadvantaging performance. To deal with high dimensional and noisy data in DTI predictions, several Deep Learning Methods have been proposed. The main disadvantages of these methods are that a great deal of training data and high computational power are required to train the complex model. Additionally, they lack the transparency in interpreting results and performance issues is a disadvantage.

Matrix Factorization Methods are another family of methods used in DTI prediction. Matrix Factorization techniques aim to find two matrices, Y_{n×k} and Z_{k×m} whose multiplication gives the interaction matrix X_{n×m} with k<<n, m. It is assumed that the drugs and targets lie in the same distance space such that the distance among drugs and targets can be used to measure the strength of their interactions. Therefore, drugs and targets can be embedded in a common low-dimensional subspace. Matrix factorization has been reported to be the most reliable methods among these DTI prediction methods based on performance. However, matrix factorization is still quite limited. For example, current techniques are unable to incorporate all available information about drugs and targets.

A persistent problem across DTI prediction methods, in particular with Matrix Factorization, is incomplete data. In other Big Data contexts, developers have attempted to address the matrix completion problem, i.e., imputing/predicting missing values of a matrix, given an incomplete matrix with values that are noisy and potentially corrupt values. A common application is the recommender systems such as the Netflix Prize. Prediction of the missing values has been an active area of research, resulting in techniques such as singular value thresholding, fixed point continuation, and matrix factorization. However, these techniques all ignore supporting data that could be integrated with the main matrix. Further still, because with matrix-based DTI prediction methods sparsely populated matrices are prevalent (e.g., drug matrices, X, target matrices, Y, and, if they exist interaction matrices M_{XY}) true drug repositioning (i.e., drug repurposing) is severely limited. There is a significant need for addressing the shortcomings of matrix-based prediction methods, in particular the matrix completeness problem.

The matrix completion problem is a particularly challenging hurdle for DTI prediction using matrix-based method is to perform the completion task of the sparse matrices of drugs, X, targets, Y , along with their interactions, M_{XY}, (shown in FIG. 1) that are central to the field of drug repositioning (a.k.a. drug repurposing).

### SUMMARY OF THE INVENTION

The present application presents techniques for predicting interactions between drugs and targets for identifying new targets for approved drugs and for identifying targets for drugs having shown no previous target efficacy. The prediction techniques herein may be used to complete incomplete (including sparse) drug-target interaction databases, for predicting drug target interactions for drug positioning. Drug positioning herein includes drug repurposing, drug repositioning, drug redirecting, and/or drug rediscovery.

In an example, the present techniques identify incomplete (e.g., missing or partially completed) entries in an interaction matrix and mines coupled matrices to develop predicted entries that are then used to complete those missing entries. In particular, in some examples, a matrix optimization function capable of simultaneous, alternating optimizations is used to mine multiple different coupled matrices, one for each dimension of the interaction matrix, to develop a predicted entry.

In another example, the present techniques are extendible to more complex associations in which incomplete entries in an interaction matrix are completed using coupled tensors to develop predicted entries. In such examples, a tensor optimization function is provided capable performing optimizations at different tensor slices which are then used to develop predicted entries for completing the interaction matrix.

The present techniques achieve novel and efficient prediction approaches for interaction matrix completion. The techniques are able to find previously unknown associations by generating a combination matrix or tensor vector of the same, while avoiding costly and laborious processes for determining interactions between matrix elements. In the drug and target context, these techniques are able to predict heretofore unknown drug-target interactions (DTIs). However, these techniques may be used in other interaction contexts in which there is an association with a population and a target. Examples of other interaction contexts include recommendation systems used by on-line streaming media content providers, such as for predicting preferences between customers and media content. That is, the present techniques may be used to improve recommendation algorithms used to provide members of a streaming service with personalized suggestions. Other example interaction contexts include financial content interactions that involve predicting a population that is associated with a target financial state. The present techniques may be used in any number of matrix and/or tensor completion in Big Data analytics, such as link prediction to identify missing probabilities in connections, urban computing to assist computing logistics applications to better understand commuting patterns, computer vision for image completion and video completion, and climate data analysis.

In an example, matrix factorization-based methods are provided that provide coupled matrix-matrix completion techniques (collectively referred to herein as “CMMC”) to complete incomplete entries of an interaction matrix by using two different input matrices each of a single dataset source. In another example, matrix factorization-based methods are provided that provide coupled tensor-matrix completion techniques (collectively referred to herein as “CTMC”) to complete incomplete entries of an interaction matrix using comprehensive information provided in input tensors or other multidimensional matrices where multiple dataset sources are used.

In an example, a computer-implemented method of performing completion of entries in a drug-target interaction matrix by predicting drug and target interactions from coupled datasets, the computer-implemented method comprises: identifying, by a computer processor, incomplete entries in the drug-target interaction matrix; accessing, by the computer processor, a drug-drug matrix and a target-target matrix, both separate from the drug-target interaction matrix; determining, by the computer processor, a matrix optimization function for use in accessing the drug-drug matrix and for use in accessing the target-target matrix; using the matrix optimization function, accessing, by the computer processor, a subset of entries in the drug-target interaction matrix, using the matrix optimization function, accessing one or more entries in the drug-drug matrix and one or more entries in the target-target matrix based on the subset of entries; performing, by the computer processor, an optimization of the matrix optimization function until a predicted interaction entry, corresponding to an entry from the drug-drug matrix and an entry from the target-target matrix, is identified for completing one of the incomplete entries of the drug-target interaction matrix and updating the drug-target interaction matrix forming an updated drug-target interaction matrix including the predicted interaction entry; and receiving, by the computer processor, subsequent drug and/or target data, comparing the subsequent drug and/or target interaction data to the updated drug-target interaction matrix and outputting one or more resulting interaction entries from the updated drug-target interaction matrix.

In accordance with another example, a computer-implemented method of performing completion of entries in a drug-target interaction matrix by predicting drug and target interactions from coupled datasets, the computer-implemented method comprises: identifying, by a computer processor, incomplete entries in the drug-target interaction matrix; accessing, by the computer processor, a drug-drug tensor and a target-target tensor; determining, by the computer processor, a tensor optimization function for use in accessing the drug-drug tensor and for use in accessing the target-target tensor; using the tensor optimization function, accessing, by the computer processor, a subset of entries in the drug-target interaction matrix and, using the tensor optimization function, accessing a plurality of slices of the drug-drug tensor and a plurality of slices of the target-target sensor; performing, by the computer processor, an optimization of the matrix optimization function for each of the plurality of slices of the drug-drug tensor and for each of the plurality of slices of the target-target tensor, thereby producing an optimum candidate drug entry and optimum candidate target entry for each slice; performing, by the computer processor, an optimization on the optimum candidate drug entries and on the optimum candidate target entries for each of the slices to identify a predicted interaction entry containing an optimum entry from the drug-drug tensor and an optimum entry from the target-target tensor, and populating one of the incomplete entries of the drug-target interaction matrix with the predicted interaction entry to form an updated drug-target interaction matrix; and receiving, by the computer processor, subsequent drug and/or target data, comparing the subsequent drug and/or target interaction data to the updated drug-target interaction matrix and outputting one or more resulting interaction entries from the updated drug-target interaction matrix.

In accordance with another example, a computer-implemented method predicting new interactions/relationships between a two sets of data based on known information, the method comprises: obtaining a first tensor of a first data set, the first tensor comprising a plurality of different slices of the first data set, each slice containing a different relationship of the first data set; obtaining a second tensor of a second data set, the second tensor comprising a plurality of slices of the second data set, each slice containing a different relationship of the second data set; analyzing an interaction matrix containing interaction data between the first data set and the second data set to identify incomplete entries in the interaction matrix; and performing an iterative minimization of an optimization function coupling the interaction matrix to the first tensor and to the second tensor to determine one or more predicted new interactions/relationships between the first data set and the second data set and updating the interaction matrix with the one or more predicted new interactions/relationships.

### BRIEF DESCRIPTION OF THE DRAWINGS

The figures described below depict various aspects of the system and methods disclosed herein. It should be understood that each figure depicts an embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

FIG. 1 is a schematic diagram of an example drug target interaction system, in accordance with an example.

FIG. 2 is a schematic illustration of an example implementation of drug-target interaction prediction system having a drug-target optimization platform, in an example.

FIG. 3 illustrates a schematic of an example in silico prediction of drug-target interaction, as may be performed by drug-target interaction system herein, in an example.

FIG. 4 illustrates various databases and database types that may be accessed by a drug-target interaction system herein, in an example.

FIG. 5 is a schematic diagram of a coupled matrix-matrix completion model operation using a matrix optimization function to complete incomplete entries in a drug-target interaction matrix, in an example.

FIG. 6 is a schematic diagram of a coupled tensor-matrix completion model using a tensor optimization function to completed incomplete entries in a drug-target interaction matrix, in an example.

FIG. 7 is a flow diagram of an example process for coupled matrix-matrix completion, in an example.

FIG. 8 is a flow diagram of an example process for coupled tensor-matrix completion, in an example.

FIG. 9 is a schematic diagram of an architecture for completing incomplete entries in an interaction matrix using coupled tensors, in accordance with an example.

FIG. 10 is a flow diagram of an example process for interaction matrix completion as may be performed by the architecture of FIG. 9, in accordance with an example.

### DETAILED DESCRIPTION

In various examples, the present application presents techniques for predicting interactions between an input populations and targeted outcomes. For example, in various examples described herein, techniques are provided for predicting interactions between drugs (example input populations) and targets (example targeted outcomes) for use in processes of new drug discovery and drug repurposing (also known as drug repositioning). In other examples, the input populations may be any suitable input population used for interaction matrix completion. That is, the present techniques may be used interaction contexts including recommendation systems used by on-line streaming media content providers, such as for predicting preferences between customers and media content. That is, the present techniques may be used to improve recommendation algorithms used to provide members of a streaming service with personalized suggestions, based on demographic and/or other identifying data on customers and target data on content available for consumption. The present techniques may be used in other interaction contexts, including financial content interactions, that involve predicting a population that is associated with a target financial state, link prediction to identify missing probabilities in connections, traffic and mobility optimization applications to better understand and optimize movement patterns of individuals, computer vision applications for image completion and video completion, and climate data analysis. As will be apparent from the examples described herein, with the present techniques, novel and efficient prediction approaches may be achieved that identify previously unknown predicted interactions between an input population and a targeted outcome, techniques that avoids costly and laborious processes typical of interaction matrix techniques, such as conventional techniques attempted for assembling drug-target interactions (DTIs) based on experiments alone.

The burgeoning fields of drug repositioning and drug repurposing present drug-target interaction prediction, especially those using matrix-based solutions, with a completion task problem. The matrix completion problem affects matrix to matrix comparisons, where matrix data is confined to particular data types, but involves matrices that are sparse or incomplete.

The matrix completion techniques herein are capable of completing one or more entries of an interaction matrix, more particularly, one or more entries of an incomplete interaction matrix. As used herein, references to an “incomplete” matrix (or “incomplete” dataset) include matrices (datasets) with one or more missing elements (also termed “entries”) or one or more unknown elements, as well as with sparse matrices (datasets). A “sparse” matrix (or dataset) is a matrix in which some number of matrix elements are zero. The number of zero-valued elements divided by the total number of elements is called the sparsity of the matrix. For example, in a drug-target interaction matrix, when all the interaction between drugs and targets are known, the interaction matrix is complete, whereas when the interaction between certain drugs and targets are unknown, that is an incomplete interaction matrix.

Conventional matrix completion problems commonly rely on experimental data to identify drug-target entries. Where there is no experimental data completing a drug-target interaction entry is fraught with error, when using conventional techniques. That completion problem is exacerbated even further, when one considers that matrices of different data types, such as drug matrices or target matrices, are themselves incomplete matrices or where different data types are in more complex configurations such as a multidimensional datasets, beyond that of the two-dimensional drug matrix of the two-dimensional target matrix.

In various examples, herein, techniques are provided that provide coupled matrix-matrix completion techniques (collectively referred to herein as “CMMC”) to complete incomplete entries of an interaction matrix by using two different input matrices each of a single dataset source. In various other examples, matrix completion techniques are provided that provide coupled tensor-matrix completion techniques (collectively referred to herein as “CTMC”) to complete incomplete entries of an interaction matrix using comprehensive information provided in input tensors or other multidimensional matrices where multiple dataset sources are used.

The present techniques achieve novel and efficient prediction approaches for interaction matrix completion. The techniques are able to find previously unknown associations by generating a combination matrix or tensor vector of the same, while avoiding costly and laborious processes for determining interactions between matrix elements. In the drug and target context, these techniques are able to predict heretofore unknown drug-target interactions (DTIs). The techniques may be used in other interaction contexts in which there is an association with a population and a target. These include media content interactions, such as between subscription customers and media content such as on-line streaming content.

In examples herein relating to drug-target interaction, “targets” and “target data” and “target information” includes proteins, enzymes, pathways, transporters, nuclear receptors, ion channels, G-protein, coupled receptors, and nucleic acids, by way of example not limitation. More generally, there are four main groups of targets frequently involved in drug-target interaction prediction, namely protein, disease, gene, and side effect. In various examples of drug-target pair prediction, matrix-matrix completion techniques and matrix-tensor completion techniques herein integrate both the chemical space of compounds (drugs) and the genomic space of targets into a unified space.

In various examples, target information may be stored in various formats including known databases, such as a protein database that includes a collection of sequences from one or more sources. Example target protein databases include, but are not limited to, GenBank, RefSeq, third party annotation (TPA), SwissProt, protein information resource (PIR), protein-RNA interaction database (PRD), protein data bank (PDB), etc. Example target gene databases herein include, but are not limited to, TARGET databases (tumor alterations relevant for genomics-driven therapy), transcription factor to target gene databases, microRNA Target Prediction databases, etc. Other example databases are described herein. Furthermore, references to target databases herein may include target data in a drug-interaction database.

Further, in various examples of drug-target interaction, “drugs” and “drug data” and “drug information” refer to therapeutics, compounds, molecular structures, combination therapies, other substances, or any chemical compound that brings about a physiological change in a target organism, such as the human body, when consumed, injected, and/or absorbed. These include chemical compounds that are generated, isolated, or discovered, and examined for the purpose of treating a condition, based on targeting of a potential target. Example drug databases are described herein. Furthermore, references to drug databases herein may include drug data in a drug-interaction database.

In various examples of drug-target interaction, the term drug-target interaction refers to the binding of a drug to a target location that results in a change the behavior and/or function of the target. Example databases are described hereinbelow. More generally, drug-target interaction includes any possible interaction between a drug and a target assessed by the coupled matrix-matrix completion (CMMC) processes and/or coupled tensor-matrix completion (CTMC) processes herein.

In some examples, the CMMC process is achieved by a matrix completion problem in which incomplete entries within an interaction matrix, M_{XY}, are coupled with additional structural information on the attributes X and Y involved in the matrix. For example, a CM MC process (e.g., implemented as a CM MC model) couples an interaction matrix, M_{XY}, with a matrix, M_{XX}, expressing functional relationships among different drugs and with a matrix, M_{YY}, expressing relationships among different targets. In some examples the CMMC process is configured to apply optimization functions for each of the matrix elements X and Y separately. These are functions that may be used to identify a population of potential targets for completing a sparse matrix entity, where an optimization may be applied to that function to select from among that population a specific population member or set of members to be used to complete the sparse matrix entity. The optimization functions may be stored in an optimization module. For example, an optimization module may include a first functional rule or set of functional rules specific to the type of element X for a M_{XX} matrix. The optimization module may include a second set functional rule or set of functional rules that are specific to the type of element Y for a M_{YY} matrix. Thus, for a drug-target interaction matrix, as the matrix M_{XY}, the matrix-matrix completion model may use a first optimization function for drug-drug matrix, M_{XX}, and a different optimization function for a target-target matrix, M_{YY}. By accessing matrices M_{XX} and M_{YY}, the CMMC process is able to identify similar drugs, similar targets, and similar drug-target interactions that satisfy optimization functions. In some examples, a matrix optimization function is used to produce one or more predicted drug-target interactions from the examined matrices, M_{XX} and M_{YY}. From these one or more predicted drug-target interactions, the CMMC process is able to then insert a predicted entry to complete the incomplete entry in matrix M_{XY}. In this way, the CMMC process replace entries that are sparse (e.g., having a zero entry value), missing (e.g., having no value), or otherwise incomplete, and thereby perform a matrix completion.

The optimization functions may analyze drugs or targets based on any number of functional relationships, including similarity between drugs, interactions between drugs, different scores for drugs, additive effects of drugs, synergistic effects of drugs, antagonistic effects of drugs, side effects of drugs, combined effects of drugs, and/or metabolism effects of drugs, among others. An optimization function is chosen corresponding to whichever relationship between matrix and tensor elements is desired, and the processes herein perform optimization on that function to identify a suitable predicted candidate. In examples herein reference is made to examining a drug-drug matrix that comprises drug similarity data. However, references herein to drug-drug similarity are intended to include any of the functional relationships herein. Similarly, references to target-target similarity are intended to include any functional relationships between targets herein.

In some examples, the CMMC process applies an optimization function that identifies drug-drug similarities from the matrix M_{XX}. In some examples, the optimization function identifies drugs that display similar pharmacological characteristics to the drug of interest, obtained from the interaction matrix. As described herein, in various examples, the drug of interest is that of particular sparse entry in the interaction matrix. In various other examples, the drug of interest is determined from a sub-matrix of drugs having similar functionality to that of the drug at the sparse entry in the interaction matrix. This sub-matrix therefore may be mapped to a drug-drug matrix using an optimization function. In some examples, the processes herein may use that identified drug for interaction matrix completion based on the hypothesis, embedded therein, that similar drugs should be similar in mechanism of action, have similar side effect and be useful in treating a similar constellation of diseases. See, e.g., Brown, Adam S., and Chirag J. Patel. “MeSHDD: literature-based drug-drug similarity for drug repositioning.” Journal of the American Medical Informatics Association 24, no. 3 (2017): 614-618.

In some examples, the optimization function analyzes both the input population dataset and the target dataset in a simultaneous, alternating manner to identify one or more candidate entries for completing an incomplete entry of the interaction matrix. For example, a multi-dimensional optimization may include embedded optimization functions for each of the different matrices and use an alternating optimization to identify the entries for completing the sparse matrix. In a coupled matrix configuration, such as CMMC, the optimization function may be a matrix optimization function having two components one for performing an optimization on each of the drug-drug matrix and the target-target matrix. In a coupled tensor configuration, such as CTMC, the optimization function may be a tensor optimization function have multiple components, of which one or more are for performing on optimization on a drug-drug tensor and one or more are for performing an optimization on the target-target tensor. A tensor optimization function may include an optimization function that operates on slices of the tensor, such that the optimization function is able to find an optimal candidate for use in completing the sparse entry and then is able to optimize among the candidates for the slices to determine a single candidate from among the slices forming the tensor.

In some examples, similarity between missing drug entries and corresponding drugs in coupled matrices or tensors is assessed using a similarity score. Example similarity scores for drugs may be based on topological structure, geometry, chemical formula, similar compounds, and etc., although any number of example drug data may be used to provide similarity scores across drugs in a drug matrix (or database)

In some examples, similarity scoring between drugs is determined by an optimization function using a Morgan Fingerprint and inverse of Jukes-Cantor distance. The optimization function may calculate similarity scores using other criteria including (but not limited to) Avalon fingerprint instead or inverse of Mahalanobis distance, Kernel alignment ant, etc.

As with drug similarity determinations, in some examples, target similarity is scored in different ways. For example, the protein sequence similarity between two targets may be determined by performing sequence alignment, Morgan fingerprint, inverse of different distances, or other models.

In some examples, an optimization module will include multiple similar optimization functions (with scoring models) for drugs and/or for targets. In some such examples, the optimization module selects from among a plurality of optimization functions (with scoring models) to identify the most applicable optimization function based on drug data, target data, the drug database M_{XX}, the target database M_{YY}, and/or the drug-target interaction database M_{XY}.

In these ways, by identifying incomplete entries in a drug-interaction database, using the CM MC process with embedded drug and target functional similarity analyzers, the present techniques provide more accurate and more complete drug-target interaction matrices. The result is a more accurate interaction matrix completion process that completes sparse drug-target interaction data, allowing for the identification of predicted drug target interactions where no previous experimental data existed, whether in silico or in vitro.

In some examples, in place of a CMMC, a coupled tensor-matrix completion (CTMC) process is developed for use with multiple input matrices, for example, with multiple different drug matrices, M_{XX}, forming a drug tensor and/or multiple different target matrices, M_{YY}, forming a target sensor. These similarity matrices for drugs, M_{XX}, and for targets, M_{YY}, are often calculated in complementary ways based on different criteria, resulting in multiple M_{XX}'s and M_{YY}'s. For instance, the drug-drug similarities can be assessed using different structural and functional characteristics and in different chemical environments. When completing the interaction matrix, M_{XY}, in these situations, instead of matrices M_{XX} and M_{YY}, the CTMC process is developed to perform matrix interaction completion using tensors (e.g., 2+n multidimensional arrays, where n is an integer, and including 3-dimensional (3D) arrays, 4-dimensional arrays, and greater) that perform multidimensional functional similarity analyses for drugs and for targets. Example input tensor expressions include drug similarity tensors, T_{XXU}, and target similarity tensors, T_{YYZ}, where U and Z are integers (1, 2, . . . , n) represent the number of different contexts or matrices for M_{XX} and M_{YY}, respectively. That when =1, the drug-drug tensor T_{XXU} is a 3D tensor, when U=2 it is a 4D tensor, and so on. The process CTMC is thus able to complete incomplete entries in a lower dimension interaction matrix, M_{XY}, by executing a tensor optimization operation on higher dimensional input tensors, T_{XXU} and T_{YYZ}.

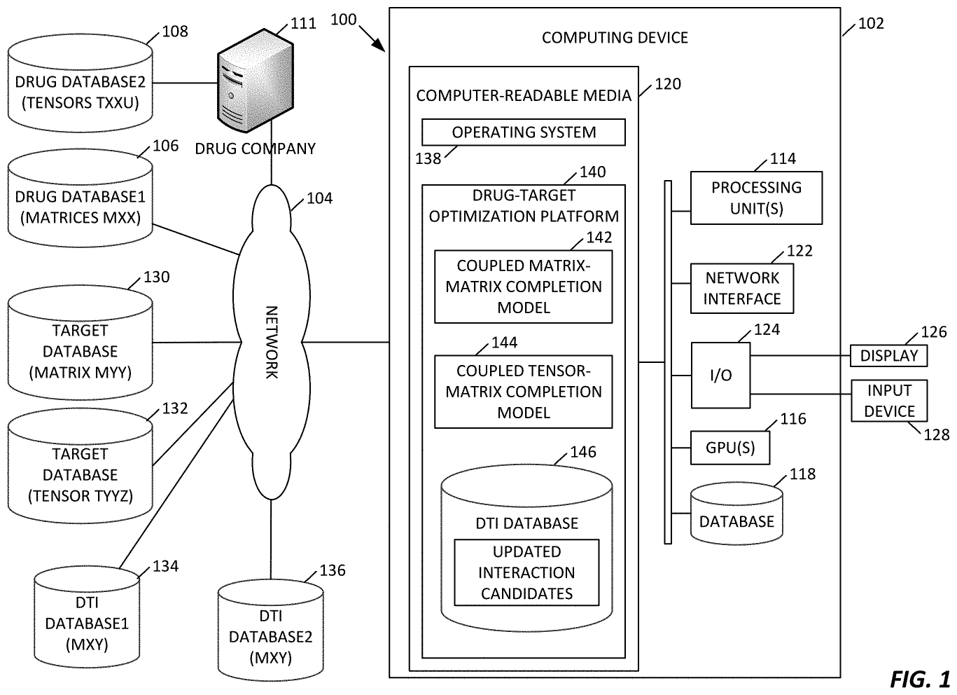

FIG. 1 illustrates an example drug-target interaction (DTI) prediction system 100 capable of determining predicted drug-target interactions and completing incomplete entries in a DTI matrix. A computing device 102 for implementing the processes described and illustrated herein is coupled to a network 104 for accessing various types of data. In the illustrated example, various databases are connected to the network 104. These databases may be network-accessible databases, each located on one or more accessible computing devices, e.g., computer servers. In some examples, one or more of these databases may be cloud-based and accessible using cloud-based access by the DTI prediction system 100.

In FIG. 1, the DTI prediction system 100 is coupled to drug databases 106 and 108 and target databases 110 and 112. Drug database 106 contains a drug-drug matrix, M_{XX}, that may contain drug similarity data for a plurality of different drugs. In some examples, the drug database 106 may include multiple different drug-drug matrices, M_{XX}, where each matrix may be different based on the drug data contained therein and/or based on the type of similarity/interaction scoring used to populate the entries in the matrix.

In the illustrated example, the drug database 108 is a private database of a drug company server 111 and accessible via an electronic request from the DTI prediction system 100 to the drug company server 111. More particularly, however, the drug database 108 differs from the drug database 106 in that the drug database 108 includes a plurality of drug-drug matrices, M_{XX}, forming a drug tensor. For example, one drug-drug matrix in the database 108 may have been formed using one specific drug database and a Morgan fingerprint form similarity model, while another drug-drug matrix may have been formed using the same drug database and an Avalon similarity model. The database 108 stores these multiple drug-drug similarity matrices configured into a drug-drug similarity tensor, T_{XXU}, formed of these multiple matrices. As the matrices, in this example, are of the same drug database but different in similarity models used to form them, the resulting tensor, T_{XXU}, would be called a 3D tensor. Another example 3D tensor would be formed from a drug-drug similarity matrix having one drug database but multiple different similarity models within that matrix. Alternatively, using the same similarity model (e.g., Morgan fingerprints) on two different drug databases, forms another slice for T_{XXU} and makes the tensor a 4th order tensor. The dimension of the drug-drug similarity tensor of database 108 could be increased further by adding distance inverses or other layers or slices to the T_{XXU} tensor. In any event, the drug-drug tensor thus has a higher dimension than any single drug-drug matrix used in forming the tensor. Thus, in some examples, the drug tensor 108 has a U dimension that represents different similarity scoring modalities between drugs. More generally, the database 108 contains drug-drug similarity information across any different number of information types. As such, the U dimension may represent any number of different types of interrelationships between drugs. Thus, the drug database 108 may represent a drug-drug matrix like that of the drug database 106, but differing in size, dimension, similarity, drugs, or different sources.

Also, as illustrated, target databases 130 and 132 are coupled to the network 104. The target database 130 contains a target-target matrix formed using a single target database and, for example, using target-specific similarity model. The target database 132, by contrast, includes plurality of different target-target matrices that have been configured into a target-target tensor, T_{YYZ}, of dimension defined by Z. In other examples, the target database 132 may represent a target-target matrix like that of the target database 130, but differing in size, dimension, similarity, targets, or different sources or any other information, similar to the differences between database 106 and 108.

Also, in the illustrated example, drug-target interaction (DTI) databases 134 and 136 are coupled to the computing device 102 through the network 104, where one or more of these databases may be incomplete databases.

While the example of FIG. 1 shows example databases, any number of different databases may be accessed by the DTI prediction system 100 through the network 104. These include databases of different types of drug-related information for use DTI predictions in silico. Generally, speaking, for DTI prediction applications, these databases can be classified into four categories, DTI databases, drug-drug databases, target-target databases, drug-target interaction databases (e.g., binding affinity databases), and supporting databases. Supporting databases may be any database containing external resources complemented with essential experimental and supporting information on genes and cellular effects. For example, CancerResource provides experimental data in addition to the information on interactions. See, e.g., Ahmed, Jessica, Thomas Mainel, Mathias Dunkel, Manuela S. Murgueitio, Robert Adams, Corinna Blasse, Andreas Eckert, Saskia Preissner, and Robert Preissner. “CancerResource: a comprehensive database of cancer-relevant proteins and compound interactions supported by experimental knowledge.” Nucleic acids research 39, no. suppl_1 (2011): D960-D967.

The DTI databases 134, 136 include databases collecting DTI information and other related information. In some examples, the DTI databases 134, 136 include databases that are unstructured, in that the data is not stored in DTI format, but where the databases contain data that can be used to generate DTI database. An example is the KEGG database which is an extensive database that covers many types of biological data from genes/proteins to biological pathways and human diseases. Within the KEGG database, two sub-databases, KEGGDRUG and KEGGBRITE contain data that can be used for DTI predictions. The ChEMBL database is also not specifically a ‘drug-target’ database, as it was established based on collecting bioactive compounds. However, combined with targets and other related biological information, the ChEMBL database can also be used in drug-target repositioning and repurposing. Similar to the ChEMBL database, the IntAct database is a database that contains molecular interactions and can be used for drug research. The LINCS database is data portal that contains biochemistry data that aims to understand changes in gene expression and cellular processes that are caused by different perturbing agents. Many of the perturbing agents used in the LINCS database are drugs, making it a useful data source for DTI prediction. Other databases included in this group are SuperTarget, Guide to PHARMACOLOGY (GtoPdb), DrugBank, Therapeutic Targets Database (TTD), STITCH, ChemProt 3.0, and DGldb 3.0.

In any event, in some examples, the DTI databases 134, 136 may represent any of a number of databases, including the following example databases and database types.

ChEMBL: The data stored in the ChEMBL database contains more than 1.9 million chemical compounds. Within these compounds, over 10 thousand drugs and more than 12 thousand targets are included in ChEMBL.

ChemProt 3.0: The ChemProt database is a disease chemical biology database that integrates data from multiple chemical-protein annotation databases and disease-associated Protein-protein interaction (PPI).

DGldb 3.0: The DGldb database integrates multiple data sources that cover information in disease-related human genes, drugs, drug interactions and potential drug ability.

DrugBank: The DrugBank database is a popular databases and has been widely used as a drug reference resource that includes both bioinformatics and cheminformatics, include detailed drug data with comprehensive drug target information.

GtoPdb: the GtoPdb database contains the ligand-activity-target relationships data that were collected from pharmacological and medicine chemistry literature.

IntAct: The IntAct database is an open source database of molecular interactions populated by data from literature and other data sources.

KEGG: The KEGG database is a comprehensive database that provides many types of knowledge about genes and genomes. The whole database can be summarized in four major categories. The first one is systems information, contains three sub-databases: KEGG PATHWAY, KEGG BRITE, and KEGG MODULE. The second category contain genomic information. In this group, four sub-databases are included: KEGG ORTHOLOGY, KEGG GENOME, KEGG GENES and KEGG SSDB. The third category holds the chemical information. Five sub-databases are in this category: KEGG COMPOUND, KEGG GLY-CAN, KEGG REACTION, KEGG RCLASS and KEGG ENZYME. The last category is health information that covers four sub-databases: KEGG DISEASE, KEGGDRUG, KEGG DGROUP and KEGG ENVIRON. The KEGG DGROUP database contains information regarding drug interaction networks including DYIs, drug metabolism and indirect interactions with enzymes and target genes.

LINCS: The LINCS database is a network-based landscape to describe how different perturbing agents influence cellular processes. In total, at present, there are 398 datasets collected in the LINCS database including fluorescence imaging, ELISA and ATAC-seq data, etc. The majority datasets (177 datasets) in LINCS are KINOMEscan kinase-small molecule binding assays. This assay is used to measure binding interactions between test compounds.

PROMISCUOUS: The PROMISCUOUS database was established in 2011 and proposed as a database for network-based drug repositioning. This database contains three different types of data: drugs, proteins and side effects. The protein data are extracted from UniProt and incorporated with the 3D structure information from Protein Data Bank (PDB). Drugs and side effects are extracted and incorporated from SuperDrug and SIDER, respectively. In addition to DTIs and drug side effects linkages, PROMISCUOUS also includes data on drug-drug similarities and PPI.

STITCH: The STITCH database stores information for inter-actions between proteins and small molecules.

SuperTarget: The SuperTarget database covers DTI information with drug metabolism, pathways and Gene Ontology (GO) terms. Medical indications and adverse drug effects are also included in this database.

D TTD: The D TTD database provides therapeutic proteins, nucleic acid targets and corresponding drug information.

Example drug databases 106, 108 and target databases 130, 132 may represent any of a number of databases, including the following example databases and database types.

In this category, six databases are included. They are BRENDA [283], PubChem [279], SuperDRUG2 [284], DrugCentral [285, 286], PDID [287], Pharos [288] and ECOdrug [289].

Among these databases, SuperDRUG2 and DrugCentral are proposed as ‘drug-centered’ (drug-drug similarity) databases. Since PubChem is a database established on collecting millions of chemical compounds, in this paper, we also list this one as a ‘drug-centered’ database. PDID and Pharos are classified as ‘target-centered’ databases. We also included BRENDA as a ‘target database’. The huge amount of enzymes and related ligands stored in BRENDA can be used as targets in DTI research. In addition, we also list ECOdrug here as a target-centered database. Different from the aforementioned ones, this database contains target information in non-human model species. Relative information can be found in Table 9.

BRENDA: The BRENDA database is comprehensive enzyme (target) database that contains 84,000 enzymes and their corresponding enzyme-ligand related information. All compounds related to enzyme catalyzed reactions are labeled as ‘ligands’ in the BRENDA database, such as substrates, products, activators, inhibitors and cofactors. In total, at present, about 205,000 enzyme ligands are collected and stored in the associated ligand database.

DrugCentral: The DrugCentral database is a drug database that contains for each drug, structure information, bioactivity and regulatory records, as well as pharmacologic actions and indications were incorporated. In this database, all drugs are simply classified into three categories, small molecule active ingredients, biological active ingredients and others.

ECOdrug: The ECOdrug database is a drug database that contains DTI data for 640 eukaryotic species. That is, the database includes drug data in a DTI database. ECOdrug contains 1194 Active Pharmaceutical Ingredients (APIs)—targeting 663 proteins to which the approved drugs bind and are responsible for the therapeutic efficacy of the drug.

PDID: The Protein-Drug Interaction Database (PDID) database is a target database that contains known protein-drug interactions and predicted protein-drug interactions for the entire structural human proteome. That is, the database includes drug data in a DTI database.

Pharos: Pharos is a platform that was established for presenting the data in the Target Central Resource Database (TCRD). The TCRD database is a comprehensive target database that contains expression data, disease and phenotype association data, bioactivity data, DTI data and databases from Harmonizome.

PubChem: The PubChem database stores information of chemical substances and corresponding biological actives. This database includes three sub-databases: Substance, Compound and BioAssay. Substance is the primary repository to store chemical information provided from individual data contributors. The Compound database contains the unique chemical structures extracted from the Substance database. All biological related data of these chemical substance data are saved in the BioAssay database.

SuperDRUG2: The SuperDRUG2 database is a drug database classified into two categories: small molecules and biological/other drugs. The database includes drugs and drug target data, as well as 2D- and 3D-structure information of small molecule drugs, drug side effects, drug-drug interactions and drug pharmacokinetic parameters.

Other types of databases may also be accessed by the DTI prediction system 100. These include binding affinity databases, e.g., that contain data on chemical-protein binding affinities. BindingDB is mainly focused on collection of binding affinity data between drugs (drug-like molecules) and target proteins. PDBbind is established based on binding affinity measurements of biomolecular complexes from PDB. PDSP Ki is similar to BindingDB, which also contains a large number of binding affinity data on DTIs. Table 10 shows the relative information of these three databases.

These databases include the BindingDB database which is a repository that contains experimental protein-small molecule interaction information. The PDBbind database includes data on protein structural information and energetic properties, as well as binding affinity data. The PDSP Ki database stores binding affinities data of drugs/chemical compounds for four different types of proteins, i.e., receptors, neurotransmitter transporters, ion channels, and enzymes.

In the example of FIG. 1, the DTI prediction system 100 is implemented on a single network accessible server. However, the functions of the DTI prediction system 100 may be implemented across distributed devices connected to one another through a communication link. In other examples, functionality of the DTI prediction system 100 may be distributed across any number of devices, including the portable personal computer, smart phone, electronic document, tablet, and desktop personal computer devices shown. In other examples, the functions of the DTI prediction system 100 may be cloud based, such as, for example one or more connected cloud CPU (s) customized to perform machine learning processes and computational techniques herein. The network 104 may be a public network such as the Internet, private network such as research institution's or corporation's private network, or any combination thereof. Networks can include, local area network (LAN), wide area network (WAN), cellular, satellite, or other network infrastructure, whether wireless or wired. The network can utilize communications protocols, including packet-based and/or datagram-based protocols such as internet protocol (IP), transmission control protocol (TCP), user datagram protocol (UDP), or other types of protocols. Moreover, the network 104 can include a number of devices that facilitate network communications and/or form a hardware basis for the networks, such as switches, routers, gateways, access points (such as a wireless access point as shown), firewalls, base stations, repeaters, backbone devices, etc.

The computing device 102 includes one or more processing units 114, one or more optional graphics processing units 116, a local database 118, a computer-readable memory 120, a network interface 122, and Input/Output (I/O) interfaces 124 connecting the computing device 102 to a display 126 and user input device 128.

The memory 120 may be a computer-readable media 120 and may include executable computer-readable code stored thereon for programming a computer (e.g., comprising a processor(s) and GPU(s)) to the techniques herein. Examples of such computer-readable storage media include a hard disk, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units of the computing device 102 may represent a CPU-type processing unit, a GPU-type processing unit, a field-programmable gate array (FPGA), another class of digital signal processor (DSP), or other hardware logic components that can be driven by a CPU.

In the illustrated example, in addition to storing operating system 138, the memory 120 stores a drug-target optimization platform 140, configured to execute various processes described and illustrated herein. In an example, the drug-target optimization platform 140 includes a coupled matrix-matrix completion model 142 for executing a CMMC process and a coupled tensor-matrix completion model 144 for executing a CTMC process, each in accordance with example techniques described herein. Additionally, the memory 120 includes a DTI database 146 updated by the models 142 and 144 and includes, in some examples, interaction candidates resulting from the processes of these models. The DTI database 146, for example, may be include updated interaction candidates determined from the completion models herein.

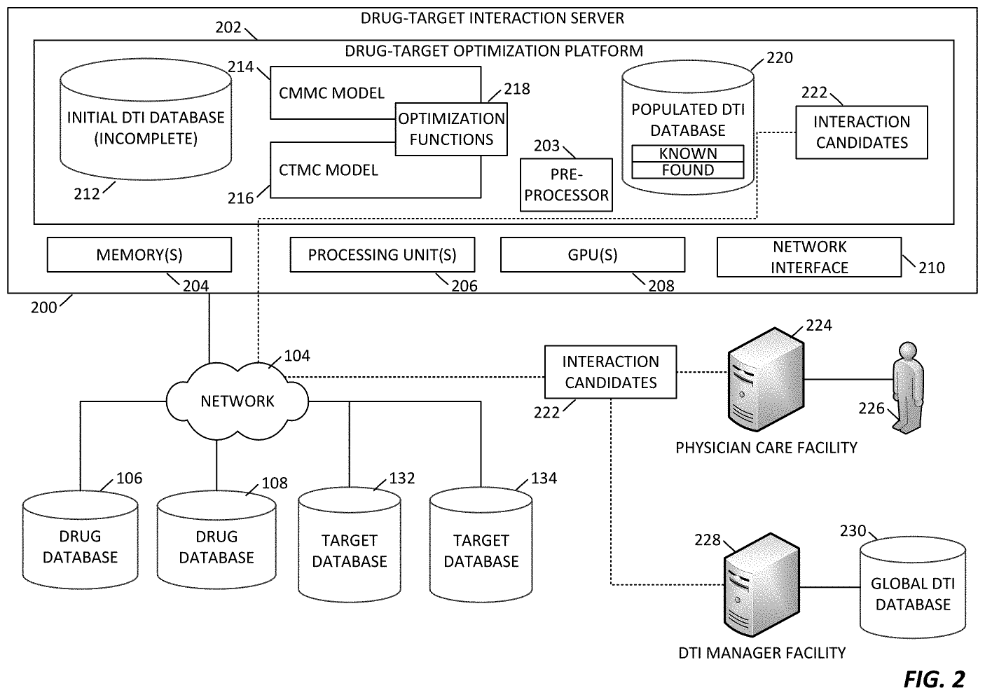

FIG. 2 illustrates an example configuration of an implementation of a DTI prediction system, such as that of FIG. 1, showing a drug-target interaction server 200 having a drug-target optimization platform 202. The drug-target interaction server 200, includes one or more memories 204, one or more processing units 206, one or more graphics processing units 208, and a network interface 210 for connecting the network 104.

The drug-target interaction server 200 is configured to access and received drug database information and target database information, perform optimization processes on that information, to update an interaction matrix comprising prediction interactions to include interaction candidates can that complete incomplete entries in the interaction matrix. In the illustrated example, the drug databases 106 and 108 provide drug-drug matrix data and/or drug-drug tensor data to the drug-target optimization platform 202 through the network 104. Similarly, target-target database 130 and target-target tensor 132 provide target data.

In an example, the drug-target optimization platform 202 receives the drug database and target database information and performs a pre-processing on the received information, using a pre-processor 203. That pre-processing may include determining the type of data in the received database, including whether the database is a drug database or a target database, for drug databases determining and target databases determining likelihood similarities. For example, it may include the process of replacing the binary values (either zero or 1) by interaction likelihood values (values between zero and 1) in the given drug-drug database or target-target database.

In an example, the pre-processing performs data normalization, for example, normalizing received drug database information from multiple different databases to remove duplication, biases, errors, overlaps, incomplete entries, etc. Similarly, normalizing may be performed on target database information, for example, to remove duplication, biases, errors, overlaps, incomplete entries, etc.

In an example, the drug-target optimization platform 202 receives the drug-drug database information and the target-target database information and generates an initial DTI database 212. In some examples, the received database information is DTI database information which is stored locally as the initial DTI database 212, that is, the DTI database 212 may be pre-existing. In either example, the initial DTI database 212 is a sparse or incomplete database, for example, one that has been generated from sparse or incomplete initial drug-drug similarity data and/or sparse or incomplete target-target similarity data.

To complete (i.e., either fully complete all incomplete entries or complete only some of the incomplete entries) the DTI database 212 with predicted drug-target interactions, the drug-target optimization platform includes a coupled matrix-matrix completion (CMMC) model 214 and a coupled tensor-matrix completion (CTMC) model 216. Each model 214 and 216 accesses one or more optimization functions 218, processed through an optimization process in either of the models 214/216, to generate predicted drug-target interactions, which are then populated into a DTI database 220. The predicted drug-target interactions are used to generate interaction candidates 222 for future treatment of a subject. In this way, and as discussed in examples herein, the drug-target optimization platform 202 is capable of performing drug-repurposing and drug-repositioning through generating the populated DTI database 220 from an initial sparse or incomplete DTI database or from initial input of drug-drug and target-target matrices to determine predicted drug-target interactions where no previous in silico or in vitro data on the interaction is known. The result is that the interaction candidates 222 will include known drugs re-positioned, i.e., identifying existing drugs that failed approval for new therapeutic indications, or re-purposed for new targets, i.e., identifying new diseases to be treated with already approved drugs.

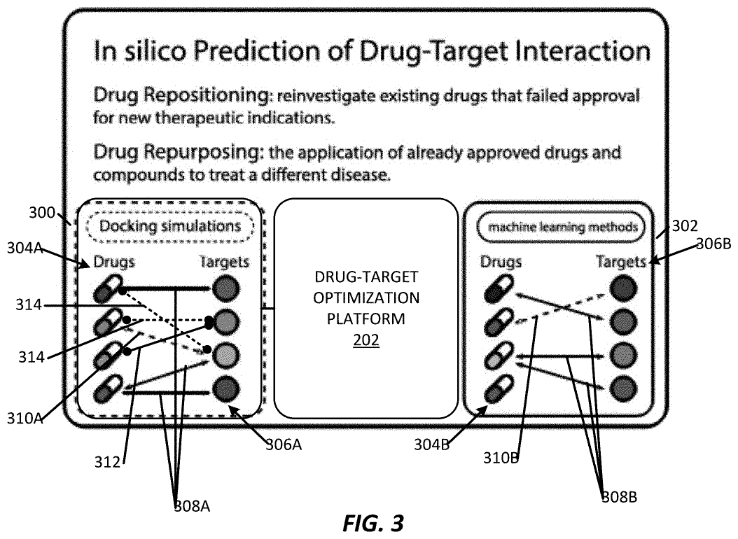

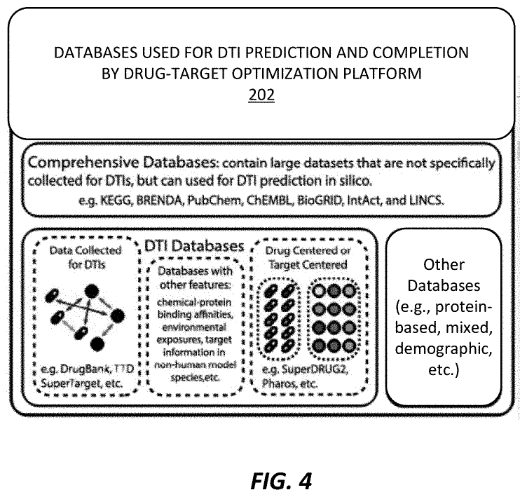

FIG. 3 illustrates an example of in silico prediction of drug-target interaction, as may be performed by drug-target interaction server 200. Two types of DTI prediction models are shown, a simulations models 300 and machine learning models 302. In the simulations model 300, various drugs 304A and targets 306A are shown having known interactions, from in vitro testing, labeled 308A and predicted interactions from conventional in silico labeled 310A. Similar labeling is used in the machine learning models 302. The drug-target optimization platform 202 has analyzed the drug-drug and target-target data contained within the simulations model 300 and identified new target indications for drugs previously having no targets, labeled 312, and identified additional targets for drugs already associated with other targets, labeled 314. FIG. 4 illustrates examples of various databases and database types that may be accessed by the drug-target optimization platform 202 in determining these new drug-target interactions 312 and 314, using the optimization techniques described herein. These include a category labeled other databases, which may be databases that are neither exclusively drug-centered nor target-centered, examples of which include protein-based databases, mixed databases containing combinations of different database types, and databases of demographic information.

Returning to FIG. 2, the drug-target optimization platform 202 includes scalable algorithm processes for coupled matrix-matrix and coupled tensor-matrix completion. These processes are applicable to the general case in which the coupled matrices/tensors are sparse themselves. Example implementations of each of the models, CMMC model 214 and CTMC model 216, are described below.

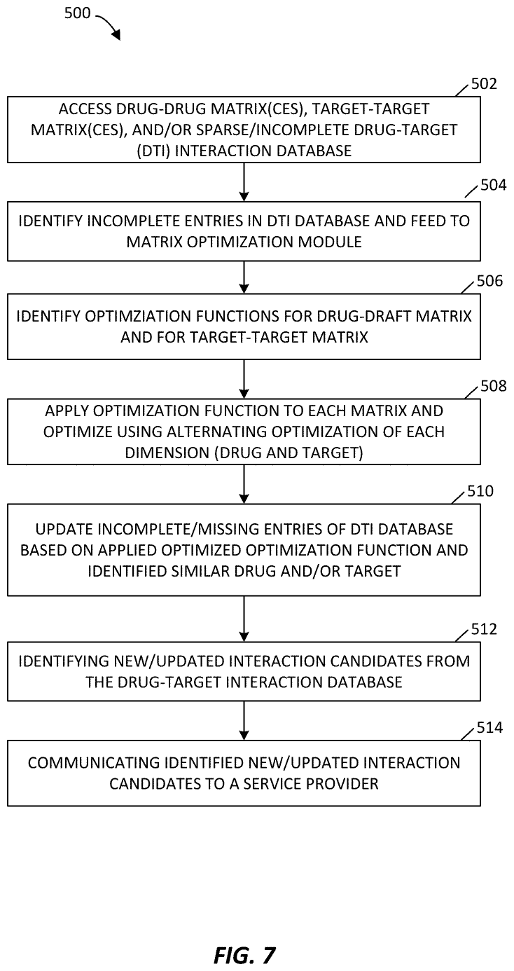

In an example, the CMMC model 214 is configured to complete incomplete entries in the DTI matrix 212. FIG. 7 illustrates a process 500 as may be performed by the CMMC model 214 in an example.

Initially, the drug-target optimization platform 202 may analyze received drug-drug matrix data (106, 108), target-target matrix data (132, 134), and/or DTI data (212), and determine which of the of the CMMC model 214 and the CTMC model 216 is to be used for completion of incomplete entries in the DTI database 212. For example, the drug-target optimization platform 202 may access the databases and analyze domain information contained in the respective database information. In some examples, the platform 202 receives data and analyzes the domain information contained in the received data. In some examples, the drug-target optimization platform 202 determines the dimensionality of the received database information and uses that to determine which model, 214 or 216, to use. In the drug-drug dataset or the target-target dataset are 3D datasets or higher, than the CTMC model 216 is used. In some examples, single drug-drug matrix and single target-target matrix databases are result in the CM MC model 214, and multiple drug-drug matrices and/or multiple target-target matrices result in the CTMC model 216. In some examples, instead of the dimensionality of drug and target databases, the determination between models is determined based on the type of data within the drug matrix database and target matrix database information. The drug-target optimization platform 202 may access or receive multiple drug-drug matrices, each having different information on a same set drugs, and, in response, determine that the CTMC model 216 is to be applied. The drug-target optimization platform 202 may make a similar determination in response to accessing or receiving target matrices having different information on the same set of targets.

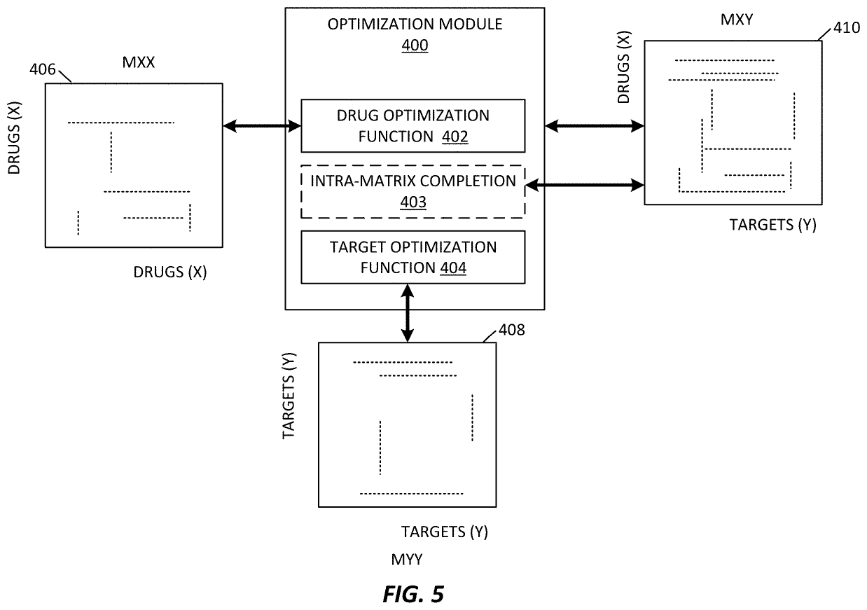

In FIG. 7, a process 502 obtains or accesses drug-drug similarity data, e.g., drug database information in a matrix, M_{XX}, as shown in FIG. 5. The process 502 obtains or accesses target-target matrix data, e.g., target database information in a matrix, M_{YY}, as shown in FIG. 5. The process 502 obtains or accesses DTI database data. In the example of FIG. 5, the process 502 accesses drug-drug similarity matrix 406, target-target similarity matrix 408, and/or drug-target interaction matrix 410. The drug-drug similarity matrix 406 may represent a matrix from the database 106 or 108, and the target-target similarity matrix 408 may represent a matrix from the database 132 or 134. The drug-target interaction matrix 410 may represent the initial incomplete database 212 that is to be used to generate a new completion matrix. The drug-target interaction matrix 410 may represent the existing populated DTI database 220 which is itself updated with completed entries as a result of processes herein.

A process 504 analyzes the DTI database and identifies entries that are incomplete, i.e., where there is absent or a zero value for a particular drug target interaction. These would be incomplete entries of the DTI matrix 410. The process 504 feeds the one or more identified entries to a optimization module 400 that includes a matrix optimization function 402 for drugs and a matrix optimization function 404 for targets.

At a process 506, an optimization function is determined for drugs and for targets. For example, the process 500 may determine a drug similarity model that may be used as a functional analyzer to identify drugs in a drug-drug matrix that are functionally similar to the drug having an incomplete target interaction in the DTI database. At the process 506, a optimization function for the drug-drug matrix 406, M_{XX}, may be determined, and a separate optimization function of the target-target matrix 408, M_{YY}, may be determined.

At a block 508, performed by the CMMC model 214, an optimization is performed on the optimization function(s) using an alternating optimization of each dimension, that is a drug specific optimization and a target specific optimization. For example, the process 508 may perform an initial optimization on the optimization function 402 for the drug-drug matrix, M_{XX}, holding the optimization function 404 for the target-target matrix, M_{YY}, fixed, and then fix the drug-drug matrix optimization function 402 and perform an optimization on the target-target matrix optimization function 404, and then repeat process in this alternating manner.

In this way, at the process 508, the CMMC model 214 is able to perform a completion process on an interaction matrix by utilizing additional meaningful and useful information on drug-drug similarity (or other factors) and target-target similarity (or other factors) by accessing separate, respective matrices and using an optimization perform interaction matrix completion. That is, the process 508, attaches two matrices (M_{XX} and M_{YY}) to an interaction matrix (M_{XY}).

In some examples, the CMMC model 214 optionally includes within the process 508 two completion stages.

In a pre-processing stage, the process 508 may use known processes to predict an unknown entry in the interaction matrix by considering entries within the matrix M_{XY} that are near the incomplete/missing entries. This is an intra-matrix completion process. WKNK is an example known process. This stage, which may be optional, may be performed by an optional intra-matrix completion process 403, as part of a pre-processing process such as performed by the pre-processor 203.

In the main completion stage, the process 508 accesses data provided in the coupled matrices M_{XX} and M_{YY}, and using a optimization, identifies similar data that is used to generate a predicted interaction thereby completing the incomplete entry in the matrix M_{XY}. For example, the process 508 may take an unknown entry “ij” in the M_{XY} matrix, identify the drug positioned in “ith” row of the M_{XX} matrix and examine, through the drug optimization function, what other drugs are similar to this drug. The process 508 can look at the “jth” column in the M_{YY} matrix and examiner what targets have interacted with drug “i” and are similar to target “j”. In some examples, such optimization functions may be performed using Eqs. 1 and 2 and through additional constraints, as discussed further below.

Thus, the process 508 may attempt the intra-matrix completion pre-processing stage first, without relying upon the drug optimization function 402 and the target optimization function 404 and respective matrices 406 and 408. In some such examples, the process 508 may assign a predictability score to the predicted result of the intra-matrix completion value obtained. If the predictability score is below a determined threshold, then the process 508 may perform a CMMC process in accordance with techniques herein.

Using the updated interaction matrix 410, a process 510 generates the populated predicted DTI database 220 by completing incomplete entries from the DTI database 212 and storing as the DTI database 220, in response to the applied optimization function. If the DTI database 220 already exists, then the process 510 is operating to update the DTI database 220, in response to the applied optimization function. In an example implementation of FIG. 5, the process 510 continually updates the interaction matrix 410, which is a low dimension matrix, a single dimension matrix in this example, by completing incomplete entries based on the drug and target data obtained from the module 400 and uses the updated matrix 410 in updating the DTI database 212. In some examples, the matrix 410 merely represents the DTI database 212. In some examples, the matrix 410 is a virtualization of all or a portion of the DTI database 212 stored in a temporary memory for faster operation before storing as data in the DTI database 212.

As a result of these CM MC completion processes, the DTI matrix 410 (and thus the DTI database 212) will now include previously-known and newly-found predicted drug-target interactions. These predictions, for example, may replace incomplete entries that previously included a zero or 1 (with 0 indicating no predicted interaction between the drug and target and with 1 indicating a predicted interaction between the drug and the target) with a value between zero and 1, based on the determined drug-target interaction data from the module 400. The new value between zero and 1 represents the percentage at which the entries are similar. In other examples, the predictions in the DTI matrix 410 are percentage likelihood of their being an interaction between the drug and the target.

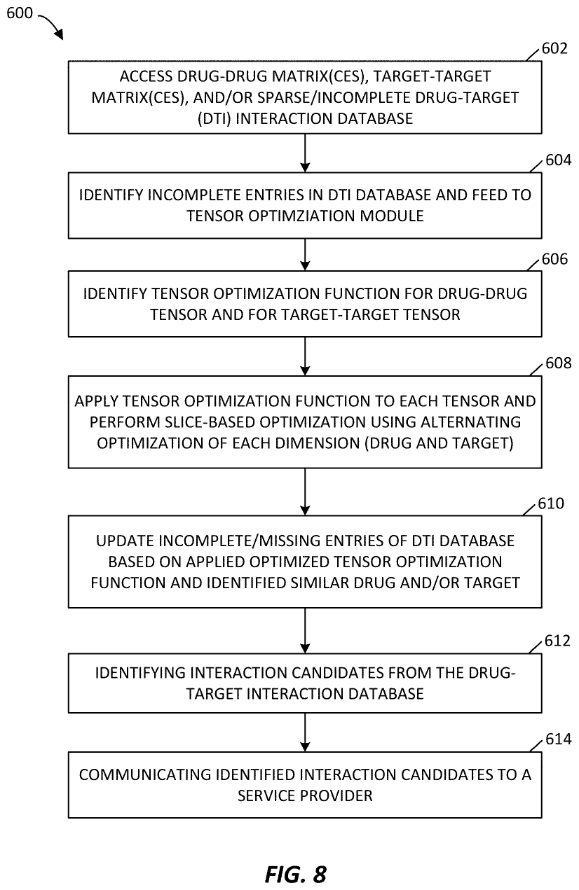

In the illustrated example of FIG. 7, with the populated DTI database 220 from process 510 having been updated with completed entries, the drug-target interaction server 200 may identify interaction candidate drugs from the database 220, where these candidate drugs are the subset of drugs that have a predicted interaction with a particular target of interest. For example, upon receiving subsequent patient data identifying a cancer type known to be associated with a particular target amenable to immunotherapy, the server 200 may examine, at process 512, the populated DTI database 220, for drugs having predicted associations with that target as indicated in the updated DTI database 220, store that information as the interaction candidates 222 and report that information, via process 514, to a physician care facility 224 for treating a patient 226 (see, FIG. 2). That is, with the updated DTI database 220, any subsequent drug and/or target data that is received may be compared to the updated DTI database 220 and an output generated indicating one or more predicted interactions from that database. In some examples, the process 514 may communicate the interaction candidates 222 to a DTI manager facility, such as another DTI server 228 for updating a global DTI database 230 with the newly predicted interactions.

Whereas the CMMC model 214 performs a interaction matrix completion using coupled matrix-matrix completion processes, the CTMC model 216 is configured to generate a low dimensional space interaction matrix of predicted drug-target interaction data from coupled tensor data. FIG. 8 illustrates a process 600 that may be performed by the CTMC model 216, in an example.

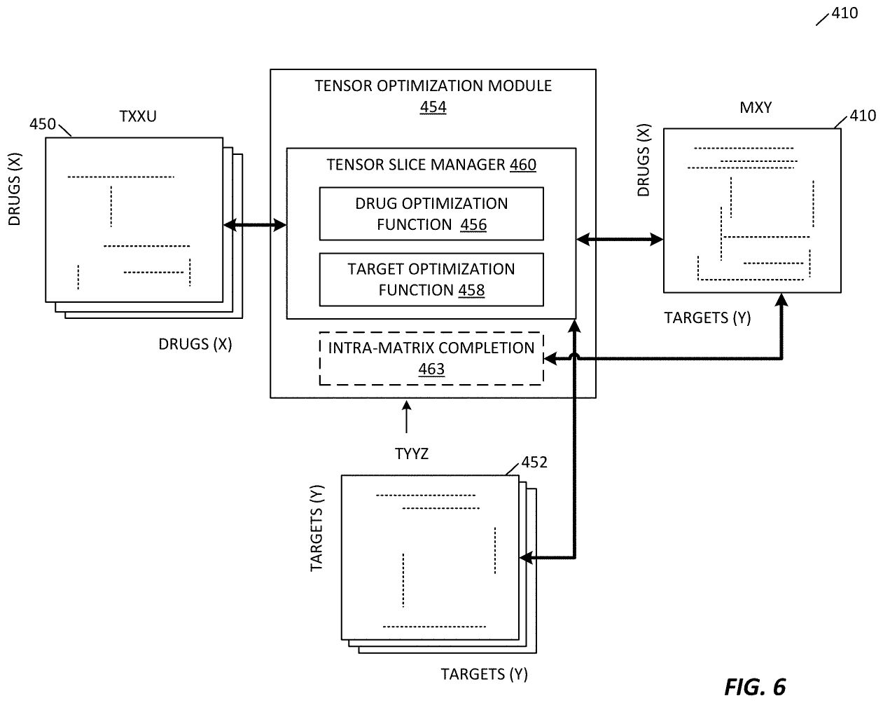

In FIG. 8, a process 602 obtains or accesses drug-drug matrix data, e.g., drug database information in a tensor, T_{XXU}, as shown in FIG. 6. The process 602 obtains or accesses target-target matrix data, e.g., target database information in a tensor, T_{YYZ}, as shown in FIG. 6. The process 602 obtains or accesses drug-target interaction database data, as well. In the example of FIG. 6, the process 602 accesses drug-drug tensor 450, target-target tensor 452, and/or the drug-target interaction matrix 410. The drug-drug tensor 450 may represent a plurality of drug-drug similarity matrices collected from the database 106 and/or 108 each matrix forming a slice of the tensor 450, and the target-target tensor 452 may represent a plurality of target-target similarity matrices collected from the database 132 and/or 134 each matrix forming a slice of the tensor 452. These tensors may include other relationship information between drugs, in addition to or instead of similarity data. Moreover, when these tensors are 3D tensors, then a tensor slice is represented by a 2D matrix of values. When these tensors have dimensions great than 3D, then a tensor slice will have a dimension higher than a 2D matrix. The drug-target interaction matrix 410 may represent the initial incomplete database 212 that is to be used to generate a new completion matrix. The drug-target interaction matrix 410 may represent the existing populated DTI database 220 which is itself updated with completed entries as a result of processes herein.

A process 604 analyzes the incomplete DTI database and identifies entries that are incomplete, i.e., where there is absent or a zero value for a particular drug target interaction. These would be incomplete entries of the DTI matrix 410. The process 604 feeds the one or more identified entries to a tensor optimization module 454 that includes a optimization function 456 for drugs and a optimization function 458 for targets. In comparison to the module 400 in FIG. 5, the module 454 is a tensor optimization module that further includes a tensor slice manager 460 that manages operation of the drug and target functions 456 and 458 to perform a optimization function and optimization for each slice of the respective tensor of across slices of the respective tensors, including in an iterative manner.

At a process 606, a tensor optimization function is determined. For example, a tensor optimization function 456 for the drug tensor, T_{XXU}, may be determined, and a separate tensor optimization function 458 for the target tensor, T_{YYZ}, may be determined.

At a block 608, performed by the CTMC model 216, an optimization is performed on the tensor optimization function(s) 456/458 using an alternating optimization of each dimension. For example, at the process 608, the tensor slice manager 460 may execute to perform an initial optimization on the optimization function 456 for the drug tensor, T_{XXU}, holding the optimization function 458 for the target tensor, T_{YYZ}, fixed, and then fix the drug tensor optimization function 456 and perform an optimization on the target tensor optimization function 458, and then repeat process in this alternating manner. The tensor slice manager 460 manages this alternative optimization process for each slice of the tensor or for one or more identified slices of the tensor that contain drug or target data similar to that of the incomplete entry identified at process 604. The tensor slice manager 460, for example, may perform a similar optimization on an slice of the drug tensor, T_{XXU}, and on a slice of the target tensor, T_{YYZ}, and then determine if the optimization is sufficient enough to identify a predicted drug-target interaction of sufficient accuracy to use in completing the incomplete entry. In another example, the tensor slice manager 460 may perform such optimization on each of the drug and target slices and take all the predict values and perform an intersection on them. In some such examples, a constraint may be applied on each slice for slice optimization and/or a constraint may be applied at the tensor level for all slices. Whichever method, the process 608 generates a predicted drug-target interaction.

In this way, at the process 608, the CTMC model 216 is able to perform a completion process on an interaction matrix by utilizing additional meaningful and useful information on drug-drug similarity (or other factors) and target-target similarity (or other factors) by accessing separate, respective tensor and using an optimization perform interaction matrix completion. That is, the process 508, attaches two tensors (T_{XXU} and T_{YYZ}) to an interaction matrix (M_{XY}).

In some examples, the CTMC model 216 optionally include a known intra-matrix optimization process to predict an unknown entry in the interaction matrix by considering entries within the matrix M_{XY} that are near the incomplete entries. This stage, which may be optional, may be performed by an optional intra-matrix completion process 463 similar to the process 403 in FIG. 5. The process 608 may optionally attempt to completion optimization using the process 463 before using the tensor slice manager 460 based on decision making like that described in reference to FIG. 5 above.

Using the updated interaction matrix 410, a process 610 generates the populated predicted DTI database 220 by completing incomplete entries from the DTI database 212 and storing as the DTI database 220, in response to the applied optimization function. If the DTI database 220 already exists, then the process 610 is operating to update the DTI database 220, in response to the applied optimization function. In an example implementation of FIG. 6, the process 610 continually updates the interaction matrix 410, which is a low dimension matrix, a single dimension matrix in this example, by completing incomplete entries based on the drug and target data obtained from the module 454 and uses the updated matrix 410 in updating the DTI database 212. In some examples, the matrix 410 merely represents the DTI database 212. In some examples, the matrix 410 is a virtualization of all or a portion of the DTI database 212 stored in a temporary memory for faster operation before storing as data in the DTI database 212.

As a result of these CTMC completion processes, the DTI matrix 410 (and thus the DTI database 212) will now include previously-known and newly-found predicted drug-target interactions. These predictions, for example, may replace incomplete entries that previously included a zero or 1 (with 0 indicating no predicted interaction between the drug and target and with 1 indicating a predicted interaction between the drug and the target) with a value between zero and 1, based on the determined drug-target interaction data from the module 454. The new value between zero and 1 represents the percentage at which the entries are similar. In other examples, the predictions in the DTI matrix 410 are percentage likelihood of their being an interaction between the drug and the target.

With DTI database 220 updated from process 610, the drug-target interaction server 200 may identify interaction candidate drugs from the database 220, where these candidate drugs are the subset of drugs that have a predicted interaction with a particular target of interest. For example, upon receiving patient data identifying a cancer type known to be associated with a particular target amenable to immunotherapy, the server 200 may examine, at process 612, the populated DTI database 220, for drugs having predicted associations with that target, store that information as the interaction candidates 222 and report that information, via process 614, to a physician care facility 224 for treating a patient 226 (see, FIG. 2). In some examples, the process 614 may communicate the interaction candidates 222 to a DTI manager facility, such as another DTI server 228 for updating a global DTI database 230 with the newly predicted interactions.

In some examples, the processes 506/508 and 606/608 of the CMMC model 214 and the CTMC model 216, respectively, are implemented as follows.

Initially, we note that a reductive group, in general, is a linear algebraic group over a field satisfying certain conditions. Let X be a real or complex vector space, then the general linear group, GL(X), and special linear group, SL(X), are reductive groups and so are the products of reductive groups. In general, GL_{d}(X) is the set of d×d invertible matrices over X, together with the matrix multiplication operation, and SL_{d}(X) is a subset of GL_{d}(X) consisting of those elements whose determinants are 1.

An n×m real matrix can be thought of as an element in

X⊗Y≅ [symbol] ^{n×m},

where X and Y are vector spaces of dimension n and m, respectively. The group GL(X)×GL(Y) acts on the space X⊗Y by

(A₁,A₂)·B=A₁BA₂^{t},

where A^{t} is the transpose of matrix A. The group GL(X)×GL(Y) acts by linear transformations, meaning that X⊗Y is a representation of the reductive group GL(X)×GL(Y). The space of symmetric n×n matrices can be identified with the space of symmetric tensors S²X⊆X®X. The group GL(X) acts on a symmetric matrix B by

A·B=ABA^{t}.

An n×m×p tensor (i.e. a multi-dimensional array) is an element in the representation X⊗Y⊗Z≅ [symbol] ^{n×m×p} of the reductive group GL(X)×GL(Y)×GL(Z), where Z≅ [symbol] ^{p}. For a coupled matrix-tensor, we get the representation

(X⊗Y)⊕(Y⊗Z⊗W),

of the group GL(X)×GL(Y)×GL(Z)×GL(W). Using the above framework, the CMMC model 214 can resolve the matrix-matrix completion problem depicted in FIG. 5, where a first input matrix, M_{XX}, is a drug-drug similarity matrix and a second input matrix, M_{YY}, is a target-target similarity matrix. The matrix-matrix completion problem may be identified with the representation

(S²X)⊕(X⊗Y)⊕(S²Y),

of the group GL(X)×GL(Y)×GL(Z)×GL(U).

FIG. 6 illustrates a tensor-matrix completion problem, where a first input tensor, T_{XXU}, is a drug-drug similarity tensor and a second input tensor, T_{YYZ}, is a target-target similarity tensor. The tensor-matrix completion problem of FIG. 6 may be represented by

(S²X⊗U)⊕(X⊗Y)⊕(S²Y⊗Z),

of the group GL(X)×GL(Y)×GL(Z)×GL(U). These reformulations of the problem induce functions with which the sparse matrices (and sparse tensors) can be optimally completed.

Most methods for matrix and tensor completion rely upon the choice of a fixed function, such as the Euclidean or nuclear norm. If there is a high correlation between the rows/columns in a matrix, or between different tensor slices, then a different function given by the data itself could be adopted. For a machine learning problem including data points in n-dimensional space, Rⁿ, Mahalanobis distance, which is computed from the covariance matrix of the data, could also be utilized. Equivalent to the Mahalanobis distance, is using the Euclidean function after a linear change of coordinates that normalizes the covariance matrix of the data to the identity. A proper action on group G could perform the change of coordinates in the vector space X such that it preserves the mathematical structure of the data. The Kempf-Ness theorem shows that there is essentially a unique change of coordinates that is optimal in a certain sense. It is known that the group G has a unique maximal compact subgroup K. The space X has some Euclidean function and without loss of generality one may assume that K is contained in the orthogonal group SO(X).

THEOREM 1. Consider the map φ: G→ [symbol] given by φ(g)=∥g·χ∥² then either φ does not have a critical point, or every critical point is a minimum and the set of critical points is a coset, Kg, for some g∈G.

The theorem implies that there is a unique optimal function, e.g., the Euclidean function after the change of coordinates given by g. The action of K does not change the function. To avoid a degenerated case, in the absence of any critical point, one may choose a slightly smaller reductive group G instead (e.g. SL, instead of GL) or utilize a regularization that is compatible with the representation theory setup. Thus, the choice of G determines the optimal function used in the matrix optimization module 400 in the CMMC architecture of FIG. 5 and the tensor optimization module 454 in the CTMC architecture of FIG. 6.

The next step is to determine the optimization function, e.g., from the optimization functions 218, for the CMMC model 214 and for the CTMC model 216.

Assuming m data points x₁, x₂, . . . , xₘ in X≅ [symbol] ⁿ with respective mean 0 and an invertible covariance matrix A, then x=(x₁, . . . xₘ)∈V=X^{m}≅ [symbol] ^{n×m} and the function φ: SL(X)→ [symbol] , defined by φ(g)=∥g·x∥² has a critical point, namely

$$G = {A}^{- \frac{1}{2}} or {A}^{- 0.5} ,$$

In this example, the optimization function is the Mahalanobis distance. However, if the data points x₁, x₂, . . . , xₘ are not thus distributed, then a better choice of G yields a more optimal optimization function. Determining an optimal choice of G for the CMMC model 214, in an example, induces function and regularization terms that are directly used in the algorithm. Given a tensor ν∈V=X⊗Y⊗Z, applying Theorem I, the CMMC model 214 can optimize

φ(g,h,k)=∥(g,h,k)·ν∥²,

for (g, h, k)∈G=SL(X)×SL(Y)×SL(Z), using alternating optimization: first optimizing for g∈SL(X) while fixing h and k, followed by optimizing h having g and k fixed, and lastly, optimizing k while fixing g and h, until the desired convergence. This is an alternating optimization. In some examples of a coupled matrix-matrix completion process described herein, where Z is 1, this reduces to a two variable alternating optimization. The function φ(g, h, k) acts on an element of the reductive group, in particular the triple (g, h, k) in this example. Each optimization step reduces to the case of m data points x₁, x₂, . . . , xₘ in X≅ [symbol] ⁿ with mean 0 and an invertible covariance matrix A, which was discussed above. It can be shown that this procedure converges to an optimal solution and in practice only a few iterations are needed.

For the CTMC model 216, there are more potential choices for G that may yield a more optimal optimization function 218. For example, G₂=SL(X)×SL(Y)×SL(Z)×SL(U) or G₃=SL(X)×SL(Y). The CTMC model 216 may determine the optimization function from among those stored in the functions 218.

We now describe application of the foregoing to examples of the CMMC model 214 and the CTMC model 216, in an example. Assuming the actual data for x∈X is not known yet y=ω(x), where ω: X→Y is a projection map, is given. In order to estimate the missing data, ∥g·x∥² is minimized over all g∈G and x∈X with the constraint ω(x)=y. However, a unique optimal solution is no longer guaranteed for this optimization, because even the low rank matrix completion problem does not always have a unique optimal solution. Therefore, as described, in some examples, the CMMC model 214 and the CTMC model 216 are configured to find an optimal g and x using an alternating optimization process. In an example, starting with the element g as the identity, a model can find x with ω(x)=y, such that ∥x∥² is minimal. An optimal g can be now found such that ∥g·x∥² is minimal, and this procedure is repeated in this alternative fashion until a desired convergence is obtained. In some cases, such as for the CTMC model 216, finding an optimal g is in itself an iterative procedure. In that case, models 214/216 can alternate a fixed number of iteration steps for g with an optimization step for x.

In order to improve the algorithms for the CMMC model 214 and the CTMC model 216 further, the models can begin by assuming that two symmetric matrices (drug and target) M_{XX}∈S²X and M_{YY}∈S²Y are given in a way that they are coupled with an incomplete interaction matrix M_{XX}∈X⊗Y, where X= [symbol] ^{nX} and Y= [symbol] ^{nY}. Without loss of generality, the matrices M_{XX} and M_{YY} are assumed to be nonnegative definite. For an incomplete initial interaction matrix, one can assume that the only known entries of M_{XY} are at positions Ω_{XY}=((i₁, j₁), (i₂, j₂), . . . , (iₖ,jₖ)). This constraint can be written as ω_{XY}(M_{XY})=ν_{XY} where ν_{XY}∈ [symbol] ^{k} is some fixed vector, and ω_{XY}: X⊗Y→ [symbol] ^{k} maps a matrix C to (Cᵢ₁, j₁, C_{i2,j2}, . . . , C_{ik,jk})^{t}. The models 214 and 216 may use the matrices M_{XX} and M_{YY} as regularization of the matrix completion problem of M_{XY}—recall for the CTMC model 216 the tensor is made up of multiple slices of M_{XX} and M_{YY}, in an example. For some fixed regularization parameters λ_{X}, λ_{Y}, the objective function (e.g., a optimization function) to minimize is defined by:

H:=∥gₓM_{XY}g_{Y}^{t}∥_{F}²²+λ_{X}Tr(g_{X}M_{XX}g_{X}^{t})+λ_{Y}Tr(g_{Y}M_{YY}g_{Y}^{t}), (1)

over all triples (g, h, M_{XY}) with g∈SLₙ, h∈SLₘ and M_{XY}∈ [symbol] ^{n×m} with the constraint

ω_{XY}(M_{XY})=ν_{XY}.

Here ∥C∥_{F}²=Tr(CC^{t}) is the square of the Frobenius norm. for some arbitrary matrix C. The constraint relates a sub-matrix of elements to vectors pointing to coupled matrices and can be used to determine either or both thereof. Thus, the optimization functions and accompanying constraint expressions can be used to complete incomplete entries in an interaction matrix using coupled input matrices, or to complete incomplete entries in coupled input matrices using an interaction matrix. The use of an optimization function with a bi-directional constraint expression allows for this bi-directional functionality that applies to any of the examples described herein. As an example, in a drug-target context, the minimization of the constraint expression, ω_{xy}(M_{XY})=ν_{XY}, can be used to determine an incomplete entry in one or both of a drug-drug matrix and a target-target matrix using a map of known sub-entries in the drug-target interaction matrix.

Thus, Eq. (1) is therefore used to identify entries in M_{XX} and M_{YY} to simultaneously predict incomplete (e.g., sparse or missing) entries in M_{XY}. In particular, the target matrix component and drug matrix component are input class members, and Eq. (1) looks to minimize the distance between the input class members of the interaction matrix M_{XY}, that is minimize the function H. Ideally, H would minimize to 0, but that rarely happens. Instead, the Eq. (1) seeks to replace incomplete entries in M_{XY} with input class members bearing the least distance using an optimization function. First term in Eq. (1) represents the Frobenius norm of the incomplete entry in the interaction matrix, M_{XY}, the second term a Trace norm optimization function for the drug-drug matrix, M_{XX}, and the third term a Trace norm optimization function for the target-target matrix, M_{YY}, such that Eq. (1) seeks to replace the Foebenius norm with Trace norm. The regularization terms parameters λ_{X}, λ_{Y} scale the two Trace norm terms.

Applied to the process 500, for example, at the block 504, a sub-matrix of known values using the constraint ω_{xy}(M_{XY})=ν_{XY}, is identified, where ω_{xy} is a mapping function that determines a sub-set of entries of the matrix, M_{XY}, for example to determine a map entries neighboring an incomplete entry, and where ν_{XY} is a k-tuple vector from the mapping function to coupled matrices.

That sub-matrix may be communicated to process 508 which applies Eq. (1) as the matrix optimization function—in this example, including both the drug optimization function and the target optimization function in a single expression). The process 508 applies Eq. (1) and the constraint to find the most similar matrix elements from M_{XX} and M_{YY} to the sub-matrix of M_{XY}, that is, defining a fixed vector used to identify candidates in M_{XX} and M_{YY}.

In some examples, the drug-drug and target-target matrices, M_{XX} and M_{YY}, respectively, may themselves be incomplete as well. Therefore in some examples, the pre-processor 203 may perform a matrix completion process to attempt to complete incomplete entries in each of these matrices. Such as a process may be optionally performed in a pre-processing stage of process 508, for example, similarly process 608 in FIG. 8. That is, in some implementations, the drug-drug matrix is used with a constraint to clean up incomplete entries and the target-target matrix is used with another constraint for the same. To perform such intra-matrix completion, the following constraints may be imposed to identify similar entries to incomplete the incomplete entries:

$$\{ \begin{matrix}{ω}_{XX} ( {M}_{xx} ) = {v}_{XX} , \\ {ω}_{YY} ( {M}_{YY} ) = {v}_{YY} .\end{matrix}$$

as well as the convex constraints which implied M_{XX} and M_{YY} being nonnegative definite. As a result, in some examples, the CMMC model 214 (as well as the CTMC model 216) can not only complete incomplete entries in the interaction matrix, M_{XY}, but first complete incomplete entries in the coupled drug-drug and target-target matrices, M_{XX} and M_{YY}.

For the CTMC model 216, the drug-drug tensor and target-target tensor, which may be obtained from several sources or generated from multiple drug-drug matrices and target-target matrices, are given by matrices T_{XXU}∈S²X⊗U of size n_{X}×n_{X}×n_{U} and T_{YYZ}∈S²Y⊗Z of size n_{y}×n_{Y}×n_{Z}. In that case there exist two additional transformations g_{U} and g_{Z} which are diagonal matrices with determinant 1 and positive entries on the diagonal. For some fixed regularization parameters λ_{X}, λ_{Y}, the objective function (e.g., a tensor optimization function) to minimize hence becomes:

$$G := {‖ {g}_{X} {M}_{X Y} {g}_{Y}^{t} ‖}_{F}^{2} + {λ}_{X} {{\sum }^{{n}_{U}}}_{i = 1} {( {g}_{U} )}_{i , i} T r ( {g}_{X} {T}_{X X U}^{i} {g}_{X}^{t} ) + {λ}_{Y} {{\sum }^{{n}_{Z}}}_{j = 1} {( {g}_{Z} )}_{j , j} T r ( {g}_{Y} {T}_{Y Y Z}^{j} {g}_{Y}^{t} ) \qquad ( 2 )$$

In this example, Eq. (2) is similar to Eq. (1), but there are many layers, and each layer is operated on using an optimization function and alternating optimization. With Eq. (2) each coupled tensor is accessed and a slice is examined as a different a layer and the minimization of Eq. (2), with the constraint term above in Eq. (1) is used and minimization is performed on that slice (layer). Note, that while the T_{XXU} is tensor, the ith component is a matrix, and U could be greater than 1, resulting in a dimensional tensor beyond three dimensions, such as 4D, 5D, etc. The Eq. (2) with constraint are applied over all triples (g, h, M_{XY}) with g∈SLₙ, h∈SLₘ and M_{XY}∈ [symbol] ^{n×m}. Moreover, the objective function [symbol] also minimizes all the number of layers added to T_{XXU}, denoted as n_{U}, and number of layers added to T_{YYZ}, denoted as n_{Z}. Here T_{XXU}ⁱ∈ [symbol] ^{nX×nX} denotes the i-th slice of the tensor T_{XXU}.

The optimizations forming the optimizations function 218 are used to balance the various sources of drug-drug and target-target interactions, and just like g_{X} and g_{Y}, g_{U} and g_{Z} are updated iteratively. As such, in some examples of the CTMC model 216, if entries of the tensors T_{XXU} and T_{YYZ} have missing entries, certain constraints may be adopted in addition to the one which assumes that all the slices are nonnegative definite and used to complete missing entries.

As described, in the CMMC model 214 and the CTMC model 216, the matrix M_{XY} represents the interaction between drugs X and targets Y, where matrix entries are typically within the interval [0, 1]. Typically, only a small percentage of the entries of matrix M_{XY} are non-zero and many are unknown. Without loss of generality, in some configurations, we assume the interaction matrix M_{XY} is symmetric, as are the drug-drug similarity matrix, M_{XX}, and the target-target similarity matrix, M_{YY}.

After coupling these matrices, an optimization function for CMMC model 214 and the CTMC model 216 is determined, such as relying upon the choice of a fixed function, such as the Euclidean function (nuclear norm). In an example, the optimal function is determined using a multidimensional iterative optimization depicted in Eq. (1).

In an example, minimizing the objective function (e.g., optimization function) given in Eq. (1) (and same for Eq. (2)) results in finding an optimal function under which the distance between interaction matrix M_{XY} and the matrix g_{X}M_{XY}g_{Y}^{t} is minimized. This also applies to two other matrices, M_{XX} and M_{XY}, as well. It is worth mentioning again that g_{X} and g_{Y} are symmetric invertible matrices whose determinants equal to 1.

With some optimization processes, the input database information provided to a drug-target interaction server will have only a small fraction of the entries in the tensors and matrices that are known. With the present techniques, the output, i.e., an interaction database with previously incomplete entries now completed, may be many times larger than the input of the known entries in input matrices/tensors. As such, dealing with the large sized tensors may become resource intensive requiring considerable memory or computational power.

Therefore, in some examples, the output and the intermediate results can be compressed configuring the optimization platform using the following. Suppose that x∈ [symbol] ^{n×m} is a matrix with missing entries and n<<m. The optimization processes of models 214 and 216, i.e., whether matrix or tensor, respectively, can be configured to assume that only the entries in the positions (i₁, j₁, . . . , (iₖ, jₖ) are known with k<<mn, evaluation of x at the positions (iₜ, jₜ) t=1, 2, . . . , k defines a map ω: [symbol] ^{n×m}→ [symbol] ^{k} where only ω(x)=y is known. The optimal solution to minimizing ∥g·x∥², where g∈SLₙ and x∈ [symbol] ^{n×m} satisfying ω(x)=y has a very special form, namely x=h·z with h∈SLₙ and z∈ [symbol] ^{n×m} with the property that the only nonzero entries of z are zᵢₜ, ƒᵢₜ, t==1, 2, . . . , k. Therefore, instead of storing the matrix x with mn entries, the models 214 and 216 may only remember the matrix h and the nonzero entries of z, a total of n²+k<<mn numbers.

### EXAMPLES

We compared example implementations of a CM MC model and a CTMC model to conventional DTI prediction methodologies.

DrugBank Example: In an example, we compared the performance of a CMMC model using the DrugBank database as an input. For performance evaluations, we consider the CMMC optimization function optimization process described above along with three convention algorithms. For every iteration, a subset of the interaction matrix, S_{XY}⊂M_{XY}, was created by randomly selecting approximately 10% of the rows and columns of M_{XY}. This resulted in a matrix, S_{XY}, of size 678×477, which corresponded to 1% of the total number of elements of M_{XY}. Next, 10% of the entries were randomly selected and replaced by 0.5, as a surrogate for a value that is neither 0 or 1, and all four algorithms (a CMMC model and three conventional processes) were used to predict those values. We then averaged the performance of all algorithms over 100 iterations.

The comparison was divided into two parts; first, we consider drug-drug and target-target binary interaction matrices coupled with the interaction matrix, M_{XY}. Table 1 represents the results. Next, the methods were compared using the coupled drug-drug and target-target similarity matrices, M_{XX}, and M_{YY}, respectively, coupled with the interaction matrix, M_{XY}. The results are shown in Table 2. The binary interaction matrices of Table 1 contain two entries, namely 0 and 1. 0 is used for no interaction and 1 is used when there is indeed an interaction. The level of interaction is not defined. The similarity matrices of Table 2, on the other hand, contain entries having similarity scores, which are any values between 0 and 1, i.e., any value in the interval [0, 1]. For example, two drugs may be similar at any level, 50%, 70% or etc., which in that case, similarity scores are defined as 0.5 and 0.7 respectively. Moreover, any drug would be 100% similar to itself, resulting in a similarity score entry of 1. The methods presented in the Tables 1 and 2 were compared based on the total runtime, AUC, F1 score, sensitivity, specificity and accuracy. The threshold columns represent the most appropriate threshold for calling a predicted value either positive or negative to optimize the F1 score calculated over the 100 iterations.

TABLE 1
Metrics of results produced by the algorithms using binary
interaction matrices obtained from Drug Bank

| | CMMC | | WKNKN + CMMC | |
|---|---|---|---|---|
| | Mean | SD | Mean | SD |
| Runtime (s) | 0.337 | 0.026 | 0.551 | 0.033 |
| AUC | 0.664 | 0.072 | 0.664 | 0.072 |
| F1 | 0.184 | 0.110 | 0.184 | 0.110 |
| Sensitivity | 0.164 | 0.085 | 0.164 | 0.085 |
| Specificity | 0.997 | 0.011 | 0.997 | 0.011 |
| | GRMF | | WKNKN + GRMF | |
| | Mean | SD | Mean | SD |
| Runtime (s) | 1302.294 | 251.657 | 1299.383 | 252.889 |
| AUC | 0.629 | 0.078 | 0.645 | 0.083 |
| F1 | 0.061 | 0.068 | 0.072 | 0.078 |
| Sensitivity | 0.120 | 0.085 | 0.114 | 0.076 |
| Specificity | 0.986 | 0.025 | 0.988 | 0.031 |
| | L2,1-GRMF | | WKNKN + L2,1-GRMF | |
| | Mean | SD | Mean | SD |
| Runtime (s) | 1288.877 | 261.152 | 1279.952 | 254.770 |
| AUC | 0.636 | 0.078 | 0.648 | 0.076 |
| F1 | 0.062 | 0.071 | 0.074 | 0.078 |
| Sensitivity | 0.117 | 0.076 | 0.104 | 0.063 |
| Specificity | 0.986 | 0.026 | 0.990 | 0.027 |
| | NRLMF | | WKNKN + NRLMF | |
| | Mean | SD | Mean | SD |
| Runtime (s) | 1.551 | 0.086 | 1.546 | 0.076 |
| AUC | 0.597 | 0.077 | 0.602 | 0.080 |
| F1 | 0.050 | 0.062 | 0.051 | 0.063 |
| Sensitivity | 0.116 | 0.079 | 0.115 | 0.090 |
| Specificity | 0.976 | 0.047 | 0.980 | 0.062 |
| | NRLMF/3 | | WKNKN + NRLMF/3 | |
| | Mean | SD | Mean | SD |
| Runtime (s) | 37.938 | 0.570 | 37.883 | 0.665 |
| AUC | 0.596 | 0.077 | 0.602 | 0.081 |
| F1 | 0.050 | 0.062 | 0.051 | 0.063 |
| Sensitivity | 0.116 | 0.079 | 0.116 | 0.090 |
| Specificity | 0.976 | 0.047 | 0.980 | 0.063 |

TABLE 2
Metrics of results produced by the algorithms using similarity matrices
obtained from DrugBank

| | Runtime (s) | | AUC | | F1 | | Sensitivity | | Specificity | |
|---|---|---|---|---|---|---|---|---|---|---|
| Algorithm | Mean | SD | Mean | SD | Mean | SD | Mean | SD | Mean | SD |
| CMMC | 0.374 | 0.088 | 0.761 | 0.078 | 0.078 | 0.060 | 0.167 | 0.101 | 0.994 | 0.014 |
| GRMF | 1289.686 | 145.483 | 0.631 | 0.079 | 0.062 | 0.069 | 0.115 | 0.079 | 0.987 | 0.023 |
| WKNKN + GRMF | 1292.219 | 142.135 | 0.650 | 0.076 | 0.075 | 0.071 | 0.116 | 0.105 | 0.985 | 0.071 |
| L21GRMF | 1290.000 | 136.695 | 0.637 | 0.078 | 0.064 | 0.072 | 0.115 | 0.073 | 0.987 | 0.026 |
| WKNKN + L21GRMF | 1289.366 | 143.215 | 0.648 | 0.081 | 0.076 | 0.076 | 0.111 | 0.078 | 0.988 | 0.040 |
| NRLMF | 1.593 | 1.106 | 0.601 | 0.079 | 0.053 | 0.061 | 0.119 | 0.096 | 0.974 | 0.072 |
| WKNKN + NRLMF | 1.582 | 0.079 | 0.615 | 0.091 | 0.062 | 0.070 | 0.127 | 0.128 | 0.973 | 0.085 |
| NRLMF/3 | 38.537 | 1.468 | 0.600 | 0.079 | 0.053 | 0.061 | 0.119 | 0.096 | 0.974 | 0.072 |
| WKNKN + NRLMF/3 | 39.080 | 2.036 | 0.615 | 0.615 | 0.062 | 0.070 | 0.127 | 0.130 | 0.973 | 0.085 |

All methods from Table 1 were tested over the same dataset with and without the pre-processing step, called WKNKN. The WKNKN pre-processing step is used to transform binary values into interaction likelihood values in the given drug-target interaction matrix. Given the drug-target interaction matrix Y∈ [symbol] ^{n×m}, where n and m denote the number of drugs and targets, respectively, the WKNKN returns the K nearest known neighbor in descending order based on their similarities to the ith drug, dᵢ, or the jth target, tⱼ. Using WKNKN allowed us to replace given binary values with an interaction likelihood value in any of the matrices. While the literature reported notable improvement in the method using the so called pre-processing step, as it is shown in Tables 1 and 2, although WKNKN improves the average value for AUC, F1 score, sensitivity and specificity as well as accuracy, it results in higher standard deviation (SD) values as well. Therefore, the pre-processing step WKNKN may also affect the robustness of convention methods. On the other hand, with the present techniques, i.e., for both CM MC and CTMC, the results were not affected by WKNKN, in these experiments. This result demonstrated that the present techniques may be implemented without processing intensive and resource taxing pre-processing steps, such as WKNKN. The advantages of the present techniques is demonstrated, in part, by conventional techniques being limited to only using known interactions given in the initial drug-target interaction matrix (e.g., initial DTI database 212), whereas drug-drug and target-target similarity/interaction matrices, M_{XX} and M_{YY}, respectively, of the present techniques converge to the completed M_{XY} matrix (e.g., the populated DTI database 220). Moreover, the pre-processing step WKNKN only affects the values that are marked 0.5 as a surrogate for the “missing” values, and hence does not affect the results for a CMMC model or CTMC model. In any event, the data from Tables 1 and 2 illustrate the robustness of the CMMC mode 214, in various examples.

The best performances in terms of AUC, F1 score, sensitivity, specificity and accuracy across different algorithms are highlighted in Tables 1 and 2, based upon which, one may observe the following:

Performance based on AUC: The average value of AUC was calculated for each method with and without employing the pre-processing step, WKNKN. The AUCs for the CMMC model are reportedly higher than all the other methods. The highest average values of AUC was calculated for the three methods based on similarity and interaction matrices were 0.637 and 0.636, respectively. These values are remarkably smaller than those of the CMMC model which were 0.761 and 0.664, respectively. A reason that using similarity matrices for both drug-drug and target-target yields a higher AUC lies in the fact that similarity matrices contain more useful information as oppose to interaction matrices which are binary and often times sparse.

Performance based on F1 scores: In terms of F1 scores, although the average scores reported for the CMMC model, using similarity and interaction matrices, correspond to small numbers, 0.078 and 0.062 respectively, they still represent higher values than those of other methods, in this example.

Performance based on sensitivity and specificity: As shown in Table 2, reported average sensitivity and specificity values for the CMMC model were recorded as 0.167 and 0.994, respectively, using similarity information; and 0.164 and 0.997 while utilizing interaction information based on Table 1. These values are higher compared to other methods even after using the pre-processing step, WKNKN.

Performance based on runtime: An advantage of the CMMC model over the others methods was the total time that it took to perform the method over the dataset. The runtime was obtained by averaging the total running time over each iteration. As shown in Tables 1 and 2 recorded runtime for the CMMC model was notably smaller than those of other methods which represents a faster process.

TTD Database example: To further evaluate the performance of an example CMMC model, we consider the TTD database. Since the CMMC model performed better using similarity scores given in Table 2 than interaction information shown in Table. 1, we consider using similarity scores in order to evaluate the performance of the CMMC model over the TTD database, specifically, the drug-drug similarity matrix, M_{XX}, and target-target similarity, M_{YY}, along with the interaction matrix, M_{XY}. The performance of the CMMC model along with conventional four other methods, GRMF, L2,1-GRMF, NRLMF, and NRLMF/3 based on the average AUC, F1 score, sensitivity and specificity over TTD dataset are shown in Table 3. Best results are marked bold. The CMMC model obtains the best results in terms of average AUC, F1 score, sensitivity and specificity compared to the other method during a shorter period of time, for this database as well.

TABLE 3
Metrics of results produced by the algorithms using similarity matrices obtained from TTD

| | Runtime (s) | | AUC | | F1 | | Sensitivity | | Specificity | |
|---|---|---|---|---|---|---|---|---|---|---|
| Algorithm | Mean | SD | Mean | SD | Mean | SD | Mean | SD | Mean | SD |
| CMMC | 3.378 | 0.307 | 0.846 | 0.037 | 0.084 | 0.070 | 0.122 | 0.093 | 0.996 | 0.008 |
| GRMF | 4176.739 | 136.152 | 0.701 | 0.064 | 0.031 | 0.029 | 0.095 | 0.067 | 0.990 | 0.013 |
| WKNKN + GRMF | 4143.534 | 120.312 | 0.683 | 0.057 | 0.083 | 0.074 | 0.091 | 0.086 | 0.991 | 0.036 |
| L21GRMF | 4148.855 | 121.843 | 0.699 | 0.064 | 0.030 | 0.031 | 0.095 | 0.071 | 0.989 | 0.018 |
| WKNKN + L21GRMF | 4156.066 | 111.406 | 0.690 | 0.057 | 0.083 | 0.076 | 0.087 | 0.071 | 0.993 | 0.025 |
| NRLMF | 4.228 | 0.639 | 0.621 | 0.066 | 0.030 | 0.025 | 0.072 | 0.057 | 0.990 | 0.019 |
| WKNKN + NRLMF | 4.579 | 0.351 | 0.651 | 0.063 | 0.054 | 0.052 | 0.077 | 0.051 | 0.996 | 0.009 |
| NRLMFβ | 65.611 | 3.659 | 0.621 | 0.066 | 0.030 | 0.025 | 0.072 | 0.057 | 0.990 | 0.019 |
| WKNKN + NRLMFβ | 67.278 | 3.810 | 0.651 | 0.063 | 0.054 | 0.052 | 0.077 | 0.051 | 0.996 | 0.009 |

### Example CTMC Model Performance Comparisons

In order to evaluate the performance of the CTMC model, multidimensional arrays of drug-drug and target-target similarity/interaction were created and the results are shown in Table 4. In order to perform the evaluation, we incorporated both similarity and interaction information between drugs and targets in order to form the drug-drug and target-target tensors, T_{XXU} and T_{YYZ}, respectively. As shown in Table 4, the CTMC model outperformed all conventional methods and, in this example, the CM MC model in terms of average AUC and sensitivity. The results in terms of F1 score, specificity and accuracy remain the same as the CMMC model, in this example, while using similarity information, as shown in Table 2. The difference was more remarkable when the similarity scores are used for coupling, most likely because the similarity matrices are rather complete, whereas the interaction matrices are sparse.

TABLE 4

| Metric of results produced by CTMC model | | | | | |
|---|---|---|---|---|---|
| using DrugBank database | | | | | |
| CTMC Method | | | | | |
| Runtime (s) | | AUC | | F1 | |
| Mean | SD | Mean | SD | Mean | SD |
| 0.513 | 0.045 | 0.775 | 0.080 | 0.169 | 0.110 |
| Sensitivity | | Specificity | | Accuracy | |
| Mean | SD | Mean | SD | Mean | SD |
| 0.169 | 0.082 | 0.997 | 0.011 | 0.997 | 0.011 |

Comparing the performance of the CMMC model (coupled with similarity matrices) and the CTMC model, shown in Table 2 and Table 4, respectively, it is notable that the CTMC model slightly outperformed the CMMC model in terms of average values of AUC, F1 score, sensitivity, specificity and accuracy, in this example.

Table 5 demonstrates the improvement of performance as more layers are added to the CTMC model. Initially, the interaction matrices are coupled for the CTMC model. This specific case is equivalent to the CMMC model as one could consider matrices as a two-way tensors. The evaluation results in comparable, albeit better, AUC's. As another layer is added in the drug-drug tensor, T_{XXU}, namely the drug-drug similarity scores from Morgan Fingerprint, the AUC improves by approximately 10% as it is shown in Table 5. Similarly, adding another layer to the target-target tensor, the AUC improves by approximately 5%. Adding the third layer to drug-drug tensor, however, does not improve the performance. It is likely due to the fact that the similarity information, calculated by different algorithms from the same database (DrugBank), does not provide any new information hence does not improve the results.

Additionally, the recorded runtime for the CTMC model, which incorporates more information and carries out more calculations, is nonetheless faster than the other algorithms.

TABLE 5
Performance of the CTMC algorithm adding slices obtained from DrugBank

| | | # of | Runtime (s) | | AUC | | F1 | | Sensitivity | | Specificity | |
|---|---|---|---|---|---|---|---|---|---|---|---|---|
| TXXU | TY Y Z | Slices | Mean | SD | Mean | SD | Mean | SD | Mean | SD | Mean | SD |
| MXX,₁ | MY Y,₁ | 2 | 0.154 | 0.013 | 0.664 | 0.072 | 0.184 | 0.110 | 0.164 | 0.085 | 0.997 | 0.011 |
| MXX,₁, MXX,₂ | MY Y,₁ | 3 | 0.171 | 0.016 | 0.723 | 0.076 | 0.179 | 0.109 | 0.168 | 0.080 | 0.995 | 0.029 |
| MXX,₁, MXX,₂ | MY _{Y,1,} MY Y,₂ | 4 | 0.180 | 0.014 | 0.778 | 0.078 | 0.180 | 0.107 | 0.164 | 0.071 | 0.998 | 0.005 |

Table 5 illustrates metrics of results produced by the CTMC model, stratified by the number of slices used in each coupled tensor. In Table 5, M_{XX,1} denotes binary drug-drug interaction matrix, M_{XX,2} represents drug-drug similarity matrix with Morgan fingerprint scores. As for the targets, MYY_{,1} denotes binary target-target interaction matrix and MYY_{,2} represents target-target similarity matrix as an inverse of Jukes-Cantor distance of amino acid sequences.

The optimal regularization parameters for λ_{X} and λ_{Y} included in Eqs. (1) and (2) are chosen based on the performance of the algorithm during the execution of CMMC and CTMC methods. In order to determine how sensitive the proposed methods are based on the changes of the arbitrary-then-fixed parameters λ_{X} and λ_{Y}, as well as studying the roles of these parameters, the results under the CMMC model have been compared from Tables 1, 2 and 3, against different variants of λ_{X} and λ_{Y}. The results are shown in Table. 6. As setting either parameters λ_{X} and λ_{Y} to zero simply means to ignore the role of one of the drug-drug or target-target matrices, it negatively impacts the performance. We have set λ_{X} and λ_{Y} to zero and the results are shown in Table. 6.

TABLE 6
AUC Results for different λ_{X} and λ_{X}

| CMMC | DrugBank | TTD |
|---|---|---|
| λ_{X} = 0.00; λ_{Y} = 1.00 | 0.6300 | 0.4902 |
| λ_{X} = 1.00; λ_{Y} = 0.00 | 0.7005 | 0.8372 |
| λ_{X} = 0.10; λ_{Y} = 0.05 | 0.7545 | 0.8445 |
| λ_{X} = 0.05; λ_{Y} = 0.10 | 0.7580 | 0.8464 |
| λ_{X} = 0.05; λ_{Y} = 0.05 | 0.7610 | 0.8460 |

While mainly depending on the nature of the database in use, specifically the drug-drug and target-target similarity/interaction matrices, M_{XX} and M_{YY}, it was found that the smaller the values of λ_{X} and λ_{Y}, the better the prediction performance, in these examples.

Thus, as provided, in various examples herein coupled matrix-matrix completion and coupled tensor-matrix completion models are provided for prediction of DT interactions. The models may be used to overcome the sparsity of the similarity/interaction matrices. Using example implementations of the present techniques, certain unknown interactions, i.e. 0's values, were replaced by the likelihood values using K nearest neighbor method. Next, experiments were performed over coupled drug-drug, drug-target and target-target matrices, considering drug-drug similarity scores and target-target interactions. The CMMC model was tested and showed considered improvement across functions using matrices including drug-drug similarity (calculated using Extended-Connectivity Fingerprint), drug-target, and target-target interactions. The CTMC model is capable of using, in addition to the matrices, extra layers for drug-drug tensors assigned to generate drug-drug interaction. That is, the CTMC model is capable of using all the information about drugs and targets formed at different levels in drug-drug and target-target tensors. For example, the CTMC model can integrate together interaction and similarity matrices and further include addition to any other information one may have. In forming the target-target tensor, we included target-target similarity scores in addition to their interactions. Both model techniques, CMMC and CTMC, showed strong ability in order to predict new, unknown drug-target interactions.

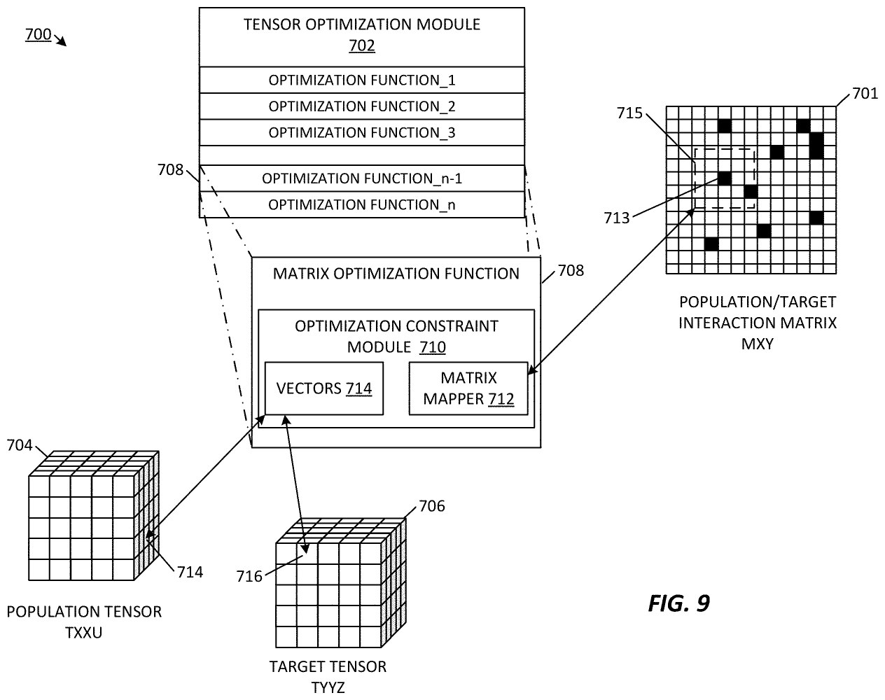

FIG. 9 illustrates an example architecture 700 for completing incomplete entries in an interaction matrix 701 using coupled tensors. The architecture 700 includes a tensor optimization module 702 having a plurality of possible optimization functions, labeled Optimization Function_1, Optimization_Function_n, each of which represents different optimization functions. These optimization functions may differ based on the functional relationships they are each designed to optimize, where those functional relationships are between populations in a population tensor 704 and/or between targets in a target tensor 706. In the illustrated example, Optimization_Function_n−1 708 is shown expanded and is a matrix optimization function, i.e., able to identify candidate entries in both the population tensor 704 and the target tensor 706. The illustrated example, the optimization function 708 includes an optimization constraint module 710 that includes a matrix mapper 712 and vectors 714. In an example implementation, the interaction matrix 701 is analyzed and an incomplete entry 713 is identified, for example, by an optimization platform within which the tensor optimization module 702 is implemented. The incomplete entry is provided to the optimization constraint module 710, and the matrix mapper 712 applies a mapping function to the interaction matrix to identify a map 715, i.e., a subset of matrix entries, in the matrix 701. The map 715 contains the incomplete entry 713 as well neighboring entries, all but one of which contain interaction matrix entries, i.e., are complete entries. The neighboring entries include adjacent and non-adjacent entries. By mapping to neighboring matrix entries to form the map 715, the matrix mapper 712 ensures that entries similar to those of the incomplete entry will be analyzed and mapped to vectors connecting to the coupled tensors 704 and 706. The shape and/or size of the map 715 may be determined by the matrix mapper 712 and may be based on various information, such as the type of data, X and Y, forming the matrix, the size of the interaction matrix, the similarly between adjacent X and/or Y entries in the matrix and any functional relationships discussed herein. In some examples, the map is symmetrical, having the same dimensions on the population axis and on the target axis. In some examples, the map is asymmetrical, with different dimensions on each axis. The map 715 may be dynamically set. For example, in some implementations, the matrix mapper 712 may apply a smaller map initially, determine if the incomplete entry 713 can be completed from vectors connecting to the smaller map without considering a larger map, and if not then the matrix mapper 712 may set a larger map and apply vectors connecting from the larger map. The matrix mapper 712 may determine the shape and/or size of the map 715 based on the sparseness of the matrix, where the more sparse the matrix is the larger the map size.

In the illustrated example, the optimization constraint module 710 receives the matrix entries contained within the map 715 and applies different vectors 714 from the map 715 to the respective population tensor 704 and the target tensor 706, according to the rules of the optimization function 708. In some examples, the optimization function 708 includes a minimization rule used to determine optimum vectors pointing to entries for use in completing the incomplete entry 713. In the illustrated example, one of the vectors 714, after the minimization rule has been applied, identifies a population entry 714 and a target entry 716 to be used to generate a completed entry for replacing the incomplete entry 713. These entries are contained in one of the slices of the respective tensors 704 and 706.

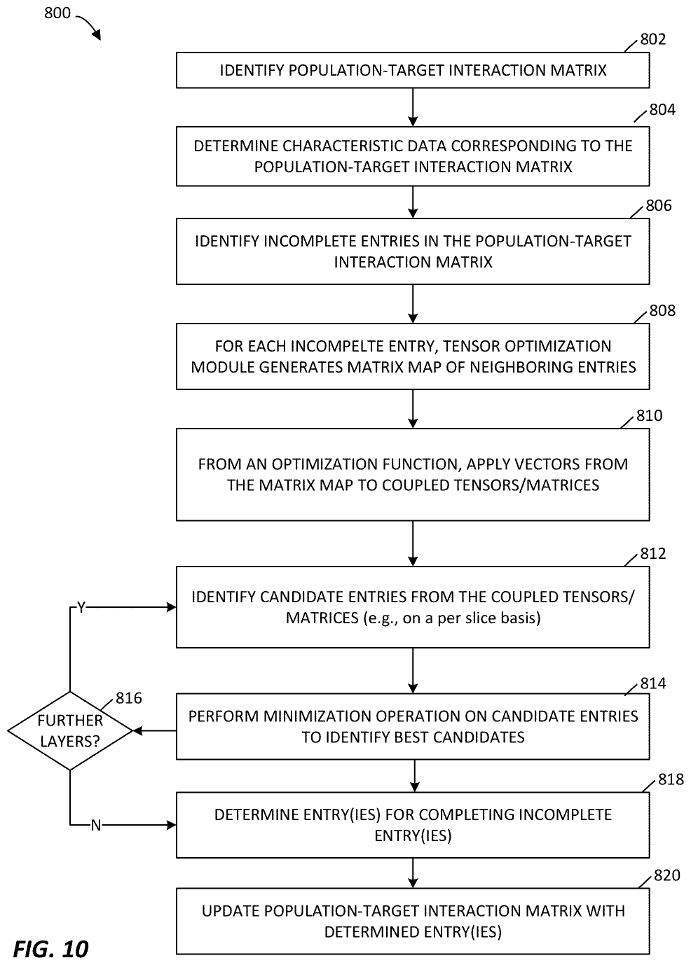

In an example, the optimization module 702, e.g., applying the optimization function 708, analyzes tensors to complete the incomplete entry 713 using a process 800 in FIG. 10. An interaction matrix is identified at block 802. At a block 804, interaction matrix data is accessed, which may include data defining the type of population data and the type of target data in the matrix, data indicating the information and/or values contained in the matrix for each of the population and target data. In the example, of a drug target interaction matrix, the population data is drug data, e.g., chemical compounds, and the stored values may be expression values corresponding to target data, which may be proteins activated/deactivated or genes expressed/non-expressed by those compounds. While not shown, in some examples, a process is performed to analyze this characteristic data and selection from a plurality of available optimization functions, an optimization function to apply to the interaction matrix. At a block 806, the tensor optimization module 702 identifies one or more incomplete entries in the interaction matrix. That is, in some examples, the process 800 operates on multiple incomplete entries simultaneously, while in some example, operations are performed on each incomplete entry in a serially manner. At a block 808, a matrix map is generated having a subset of neighboring matrix entries that are similar in population, target, or both to the incomplete entry(ies). The matrix map may be defined by the optimization constraint module 710, which applies vectors from the map to a layer of a population tensor and a target tensor respectively, at a block 810. At a block 812, the optimization constraint module 710, with the optimization function applied to the vectors and the matrix map, identifies candidate entries in each slice, e.g., a candidate population entry for the population tensor slice and a candidate target entry for the target tensor slice. In examples where the tensors are 3D data structures, a slice would be a 2D matrix. in examples where the tensors are 4D data structures, a slice would be a 3D data structure. And so on. At a block 814, a minimization is applied to the candidates according to the optimization function, the minimization identifying a best tensor entry candidate for each vector. Examples minimizations are described herein and may include a shortest distance on each vector, for example. Further, in some examples, the minimization at block 814 is an alternating minimization coordinated across the coupled matrixes, e.g., alternatingly fixing one vector while attempting to minimize the other vector and alternating this process until no further minimization can be performed. At a block 816, the process 800 determines if there are additional layers, where if so, control is passed back to block 812 to perform an optimization on a next slice of each tensor. If there are no additional layers or if the coupled datasets were coupled matrices and not coupled tensors, then control passes to block 818 to determine the population and target values for completing the incomplete entry. In the example of coupled tensors, the block 818 may apply an optimization function process, such as vector distance minimization, on the entries from each of the slices to identify the entries to use in completing the incomplete entry. At a block 820, the incomplete entry is filled in the interaction matrix by the optimization constraint module 710. The process 800 may execute again for the next incomplete entry in the interaction matrix or, as mentioned, in some examples, the foregoing processes may be performed on multiple incomplete entries simultaneously.

As mentioned the present techniques may be implemented in any number of different applications, of which a few additional examples are described.

### Example On-Line Streaming Media Content

On-line streaming media content providers continue to search for systems to better target their media content (movies, television shows, short form videos, audio such as podcasts, advertisements, and the like) to their customers. In example implementations of the matrix completion techniques herein, such content providers are able to take various interaction matrices and perform matrix completion. An interaction matrix may include population data in the form of customer demographic data (age, gender, ethnicity, location, occupation, identified interests, marital status, family status, nationality, religion, etc.), other person characteristics defined by the platform, viewing history, viewer generated reviews, third party data on customers, etc. As new customers and new population data for these customers are on-boarded, the media content provider desires to better predict which target media content will best match the population. For these new associations, where no interaction data is available in the interaction matrix, the present techniques may use optimization functions and minimization techniques to identify entry data from coupled input population and target matrices or input population and target tensors. Different optimization functions may be used and multiple different functions may be used to generate different completed interaction matrices for testing by the content provider. Optimization functions may be based on developing sub-maps of neighboring completed entries and performing vector minimization between these sub-maps and the coupled matrices/tensors. For example, sub-maps of neighboring populations with already determined interactions scores with content may be used with the techniques herein to complete incomplete interaction matrix entries.

### Service Provides (Financial Services, Transportation Services,

The present techniques may be used by financial services platforms and transportation services platforms (such as ride-hailing, food delivery package delivery, couriers, etc.) to better target services to customers. Services may include different types of financial services for customers, various types of transportation services, rates for such services, packaging of such services to offer to customers, commuting patterns for individuals or groups of individuals, etc. In example implementations of the matrix completion techniques herein, such service platforms are able to take various interaction matrices and perform matrix completion. An interaction matrix may include population data in the form of customer demographic data (age, gender, ethnicity, location, occupation, identified interests, marital status, family status, nationality, religion, etc.), other person characteristics defined by the platform, transaction history, third party data on customers, etc. As new customers and new population data for these customers are on-boarded, the service providers desire to better predict which target services will best match a customer. For these new associations, where no interaction data is available in the interaction matrix, the present techniques may use optimization functions and minimization techniques to identify entry data from coupled input population and target matrices or input population and target tensors. Different optimization functions may be used and, in some examples, multiple different functions may be used to generate different completed interaction matrices for testing by the service provider. Optimization functions may be based on developing sub-maps of neighboring completed entries and performing vector minimization between these sub-maps and the coupled matrices/tensors. For example, sub-maps of neighboring populations with already determined interactions scores with services may be used with the techniques herein to complete incomplete interaction matrix entries.

### Other Data Completion Operations (Big Data Analytics, Computer Vision, Climate Data Analysis, etc.)

The present techniques may be used to perform big data completion in other examples, such as completing images or video in computer vision applications, completing climate, geographic, demographic, and other data used in climate data applications, and other big data functions. In example implementations of the matrix completion techniques herein, these applications are able to take various interaction matrices and perform matrix completion. As new first data and/or second data is obtained, where no interaction data is available in the interaction matrix, the present techniques may use optimization functions and minimization techniques to identify entry data from coupled first data matrices and second data matrices or first data tensors and second data tensors. Different optimization functions may be used and, in some examples, multiple different functions may be used to generate different completed interaction matrices for testing by the service provider. Optimization functions may be based on developing sub-maps of neighboring completed entries and performing vector minimization between these sub-maps and the coupled matrices/tensors. For example, sub-maps of neighboring first data entries with already determined interactions scores with second data may be used with the techniques herein to complete incomplete interaction matrix entries.

### Healthcare, Bioinformatics and Medical Applications

The present techniques may be used in healthcare, bioinformatics, and medical applications beyond those described in examples above. Medical images, e.g., magnetic resonance imaging (MRI) images and computed tomography (CT) scans, are used in various healthcare care bioinformatics applications. Medical images often involve fast acquisition times, variations in imaging equipment and examination procedures, and variability due to patient movement. Any of these can lead to medical images formed of image datasets that are often incomplete. In example implementations, the matrix completion techniques herein may be used to complete incomplete portions of these medical images by treating the medical image of a 2D matrix and coupling thereto, using optimization functions, input 2D medical images. In some examples, these input 2D images may be complete images of the same region of interest (same tissue, same organ, same region of the body, etc.). In some examples, these input 2D images may be previous medical images captured for the subject, for example, at a previous point in time. In some examples, these input 2D images may be medical images captured at generally the same time as the incomplete medical image, for example, the input 2D images may be tomographic images captured at different slice depths used for generating 3D CT images. By using the optimization functions herein, similar imaging regions in input 2D images may be identified, through a minimization process, and the imaging data from these regions may be used to complete incomplete images in the captured medical image. A map of sub-pixels neighboring the incomplete portions of a medical image and vectors to coupled input 2D images may be used with optimization function and the constraint expression therein to identify one or more pixels to be used in completing the incomplete ports of the medical image. In other examples, 2D medical image completion can be performed using the techniques herein applied to coupled 3D medical images, where the 3D medical images are formed of a series of 2D slice images thereby operating as a coupled tensor. To complete the incomplete regions of a the 2D medical image, an constraint minimization may be performed on each slice of a 3D image and the optimum slice used to complete the 2D medical image. The completion may be achieved by using optimization functions based on any number of imaging features (contrast, density, pixel intensity, pixel capture color, edge detection, gradient, resolution, etc.).

The completion techniques may extend to other types of datasets and analytics used in bioinformatics pipelines and healthcare applications, where incomplete data exists. These include completing incomplete data in medical records matrices by coupling to matrices of related information, such as medical records of other patients. Other dataset examples include completing incomplete gene sequencing data, such as incomplete RNA sequencing data taken from patient samples, by coupling with datasets of other RNA sequencing data from the same tissue type, same patient, etc. Other dataset examples include performing completion dataset processes on clinical analysis data using coupled heterogeneous electronic health records (EHRs), thereby generating meaningful predicted clinical assessments when such data does not exist or exists in an incomplete manner. In other examples, the incomplete dataset may be multi-channel EEG (electroencephalogram) signal data, where missing data is encountered due to disconnections of electrodes. EEG data may be stored in matrix form. By coupling completed EEG signal data, for example, in the form of coupled matrices or coupled tensors, the optimization functions herein may be used to complete the incomplete EEG signal data.

In any of these examples, when using coupled tensors, those tensors may include the underlying dataset type of the incomplete dataset, other measured data, demographic data, or any other medical records data across the different tensor slices.

The following list of aspects reflects a variety of the embodiments explicitly contemplated by the present disclosure. Those of ordinary skill in the art will readily appreciate that the aspects below are neither limiting of the embodiments disclosed herein, nor exhaustive of all of the embodiments conceivable from the disclosure above, but are instead meant to be exemplary in nature.

Aspect 1. A computer-implemented method of performing completion of entries in a drug-target interaction matrix by predicting drug and target interactions from coupled datasets, the computer-implemented method comprising: identifying, by a computer processor, incomplete entries in the drug-target interaction matrix; accessing, by the computer processor, a drug-drug matrix and a target-target matrix, both separate from the drug-target interaction matrix; determining, by the computer processor, a matrix optimization function for use in accessing the drug-drug matrix and for use in accessing the target-target matrix; using the matrix optimization function, accessing, by the computer processor, a subset of entries in the drug-target interaction matrix, using the matrix optimization function, accessing one or more entries in the drug-drug matrix and one or more entries in the target-target matrix based on the subset of entries; performing, by the computer processor, an optimization of the matrix optimization function until a predicted interaction entry, corresponding to an entry from the drug-drug matrix and an entry from the target-target matrix, is identified for completing one of the incomplete entries of the drug-target interaction matrix and updating the drug-target interaction matrix forming an updated drug-target interaction matrix including the predicted interaction entry; and receiving, by the computer processor, subsequent drug and/or target data, comparing the subsequent drug and/or target interaction data to the updated drug-target interaction matrix and outputting one or more resulting interaction entries from the updated drug-target interaction matrix.

Aspect 2. The computer-implemented method of Aspect 1, wherein performing the optimization of the matrix optimization function comprises: performing, by the computer processor, an alternating optimization between the drug-drug matrix and the target-target matrix until the predicted interaction entry is identified, wherein the alternating optimization comprises alternatively (i) fixing a drug-drug matrix optimization while minimizing a target-target matrix optimization and (ii) fixing the target-target optimization while minimizing the drug-drug matrix optimization.

Aspect 3. The computer-implemented method of Aspect 1, wherein the drug-target interaction matrix comprises entries indicating a binding of a drug entry to a target entry that results in a change in the behavior and/or function of the target entry.

Aspect 4. The computer-implemented method of Aspect 1, wherein each target entry in the target-target matrix identifies a protein, enzyme, pathway, transporter, nuclear receptor, ion channel, G-protein, coupled receptor, or nucleic acid.

Aspect 5. The computer-implemented method of Aspect 1, wherein each target entry in the target-target matrix identifies a protein, a disease, a gene, or a side effect.

Aspect 6. The computer-implemented method of Aspect 1, wherein the matrix optimization function is a fixed function.

Aspect 7. The computer-implemented method of Aspect 1, wherein the fixed function is a

Euclidean function or nuclear norm.

Aspect 8. The computer-implemented method of Aspect 1, wherein the matrix optimization function is selected from the group consisting of a Mahalanobis distance, a Kempf-Ness function, a Morgan Fingerprint, an inverse of Jukes-Cantor distance, an Avalon fingerprint, an inverse of Mahalanobis distance, and a Kernel alignment ant.

Aspect 9. The computer-implemented method of Aspect 1, wherein the matrix optimization function is:

H:=∥gₓM_{XY}g_{Y}^{t}∥_{F}²²+λ_{X}Tr(g_{X}M_{XX}g_{X}^{t})+λ_{Y}Tr(g_{Y}M_{YY}g_{Y}^{t}),

and the optimization constraint is:

w_{xy}(M_{XY})=ν_{XY}

where M_{XY} is the drug-target interaction matrix and w_{xy} is a projection mapping operation and ν_{xy} is a two-dimensional vector, having a vector component for the drug-drug matrix and a vector component for the target-target matrix.

Aspect 10. The method of Aspect 1, the method further comprising performing the optimization of the matrix optimization function until the interaction candidate entry is identified comprises: minimizing a first distance between an entry in the drug-drug matrix to a drug in the drug-target interaction matrix, the first distance being measured according to the drug optimization function and/or minimizing a second distance between an entry in the target-target matrix to a target in the drug-target interaction matrix, the second distance being measured according to the target optimization function.

Aspect 11. The method of Aspect 1, wherein the drug-drug matrix is a drug similarity matrix.

Aspect 12. The method of Aspect 1, wherein the drug-drug matrix is a drug interaction matrix.

Aspect 13. The method of Aspect 1, wherein the target-target matrix is a target similarity matrix.

Aspect 14. The method of Aspect 1, wherein the target-target matrix is a target interaction matrix.

Aspect 15. The method of Aspect 1, wherein at least one of the drug-drug matrix and the target-target matrix is an incomplete or sparse matrix.

Aspect 16. The method of Aspect 1, wherein the matrix optimization function is scalable to be applied to a plurality of drug-drug matrices and to a plurality target-target matrices.

Aspect 17. The method of Aspect 1, wherein identifying the sparse or incomplete entries in the drug-target interaction matrix comprises identifying entries having a [0, 1] value.

Aspect 18. A computer-implemented method of performing completion of entries in a drug-target interaction matrix by predicting drug and target interactions from coupled datasets, the computer-implemented method comprising: identifying, by a computer processor, incomplete entries in the drug-target interaction matrix; accessing, by the computer processor, a drug-drug tensor and a target-target tensor; determining, by the computer processor, a tensor optimization function for use in accessing the drug-drug tensor and for use in accessing the target-target tensor; using the tensor optimization function, accessing, by the computer processor, a subset of entries in the drug-target interaction matrix and, using the tensor optimization function, accessing a plurality of slices of the drug-drug tensor and a plurality of slices of the target-target sensor; performing, by the computer processor, an optimization of the matrix optimization function for each of the plurality of slices of the drug-drug tensor and for each of the plurality of slices of the target-target tensor, thereby producing an optimum candidate drug entry and optimum candidate target entry for each slice; performing, by the computer processor, an optimization on the optimum candidate drug entries and on the optimum candidate target entries for each of the slices to identify a predicted interaction entry containing an optimum entry from the drug-drug tensor and an optimum entry from the target-target tensor, and populating one of the incomplete entries of the drug-target interaction matrix with the predicted interaction entry to form an updated drug-target interaction matrix; and receiving, by the computer processor, subsequent drug and/or target data, comparing the subsequent drug and/or target interaction data to the updated drug-target interaction matrix and outputting one or more resulting interaction entries from the updated drug-target interaction matrix.

Aspect 19. The computer-implemented method of Aspect 18, wherein the drug-drug tensor comprises a plurality of slices of drug-drug correlation data each differing in drug similarities, drug structure, drug functional characteristic, chemical environment response, drug interaction, or drug topology.

Aspect 20. The computer-implemented method of Aspect 18, wherein the target-target tensor comprises a plurality of slices of target-target correlation data each differing in binary interaction, similarity score, or target type.

Aspect 21. The computer-implemented method of Aspect 18, wherein the optimization expression is:

$$G := {‖ {g}_{X} {M}_{X Y} {g}_{Y}^{t} ‖}_{F}^{2} + {λ}_{X} {{\sum }^{{n}_{U}}}_{i = 1} {( {g}_{U} )}_{i , i} T r ( {g}_{X} {T}_{X X U}^{i} {g}_{X}^{t} ) + {λ}_{Y} {{\sum }^{{n}_{Z}}}_{j = 1} {( {g}_{Z} )}_{j , j} T r ( {g}_{Y} {T}_{Y Y Z}^{j} {g}_{Y}^{t} )$$

and an optimization constraint is:

w_{xy}(M_{XY})=ν_{XY}

where M_{XY} is the drug-target interaction matrix and w_{xy} is a projection mapping operation and ν_{xy} is a two-dimensional vector, having a vector component for the drug-drug matrix and a vector component for the target-target matrix.

Aspect 22. The computer-implemented method of Aspect 18, wherein the tensor optimization function is a fixed function.

Aspect 23. The computer-implemented method of Aspect 18, wherein the tensor optimization function is a Euclidean function or nuclear norm.

Aspect 24. The computer-implemented method of Aspect 18, wherein the tensor optimization function is selected from the group consisting of a Mahalanobis distance, a Kempf-Ness function, a Morgan Fingerprint, an inverse of Jukes-Cantor distance, an Avalon fingerprint, an inverse of

Mahalanobis distance, and a Kernel alignment ant.

Aspect 25. A computer-implemented method predicting new interactions/relationships between a two sets of data based on known information, the method comprising: obtaining a first tensor of a first data set, the first tensor comprising a plurality of different slices of the first data set, each slice containing a different relationship of the first data set; obtaining a second tensor of a second data set, the second tensor comprising a plurality of slices of the second data set, each slice containing a different relationship of the second data set; analyzing an interaction matrix containing interaction data between the first data set and the second data set to identify incomplete entries in the interaction matrix; and performing an iterative minimization of an optimization function coupling the interaction matrix to the first tensor and to the second tensor to determine one or more predicted new interactions/relationships between the first data set and the second data set and updating the interaction matrix with the one or more predicted new interactions/relationships.

Aspect 26. The computer-implemented method of Aspect 25, wherein the first data set comprises customer data and the second data set comprises financial services.

Aspect 27. The computer-implemented method of Aspect 25, wherein the first data set comprises customer data and the second data set comprises on-streaming media content.

Aspect 28. The computer-implemented method of Aspect 25, wherein the first data set comprises drug interaction data and second data set comprises drug target data.

Aspect 29. The computer-implemented method of Aspect 25, wherein the first tensor and the second tensor are each 3D tensors.

Aspect 30. The computer-implemented method of Aspect 25, wherein the first tensor and the second tensor each have dimensions great than 3D.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the target matter herein.

Additionally, certain embodiments are described herein as including logic or a number of routines, subroutines, applications, or instructions. These may constitute either software (e.g., code embodied on a non-transitory, machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

While the present invention has been described with reference to specific examples, which are intended to be illustrative only and not to be limiting of the invention, it will be apparent to those of ordinary skill in the art that changes, additions and/or deletions may be made to the disclosed embodiments without departing from the spirit and scope of the invention.

The foregoing description is given for clearness of understanding; and no unnecessary limitations should be understood therefrom, as modifications within the scope of the invention may be apparent to those having ordinary skill in the art.

What is claimed:

1. A computer-implemented method of performing completion of entries in a drug-target interaction matrix by predicting drug and target interactions from coupled datasets, the computer-implemented method comprising:
identifying, by a computer processor, incomplete entries in the drug-target interaction matrix;
accessing, by the computer processor, a drug-drug matrix and a target-target matrix, both separate from the drug-target interaction matrix;
determining, by the computer processor, a matrix optimization function for use in accessing the drug-drug matrix and for use in accessing the target-target matrix;
using the matrix optimization function , accessing, by the computer processor, a subset of entries in the drug-target interaction matrix, using the matrix optimization function, accessing one or more entries in the drug-drug matrix and one or more entries in the target-target matrix based on the subset of entries;
performing, by the computer processor, an optimization of the matrix optimization function until a predicted interaction entry, corresponding to an entry from the drug-drug matrix and an entry from the target-target matrix, is identified for completing one of the incomplete entries of the drug-target interaction matrix and updating the drug-target interaction matrix forming an updated drug-target interaction matrix including the predicted interaction entry; and
receiving, by the computer processor, subsequent drug and/or target data, comparing the subsequent drug and/or target interaction data to the updated drug-target interaction matrix and outputting one or more resulting interaction entries from the updated drug-target interaction matrix.

2. The computer-implemented method of claim 1, wherein performing the optimization of the matrix optimization function comprises:
performing, by the computer processor, an alternating optimization between the drug-drug matrix and the target-target matrix until the predicted interaction entry is identified, wherein the alternating optimization comprises alternatively (i) fixing a drug-drug matrix optimization while minimizing a target-target matrix optimization and (ii) fixing the target-target optimization while minimizing the drug-drug matrix optimization.

3. The computer-implemented method of claim 1, wherein the drug-target interaction matrix comprises entries indicating a binding of a drug entry to a target entry that results in a change in the behavior and/or function of the target entry.

4. The computer-implemented method of claim 1, wherein each target entry in the target-target matrix identifies a protein, enzyme, pathway, transporter, nuclear receptor, ion channel, G-protein, coupled receptor, or nucleic acid.

5. The computer-implemented method of claim 1, wherein each target entry in the target-target matrix identifies a protein, a disease, a gene, or a side effect.

6. The computer-implemented method of claim 1, wherein the matrix optimization function is a fixed function.

7. The computer-implemented method of claim 1, wherein the fixed function is a Euclidean function or nuclear norm.

8. The computer-implemented method of claim 1, wherein the matrix optimization function is selected from the group consisting of a Mahalanobis distance, a Kempf-Ness function, a Morgan Fingerprint, an inverse of Jukes-Cantor distance, an Avalon fingerprint, an inverse of Mahalanobis distance, and a Kernel alignment ant.

9. The computer-implemented method of claim 1, wherein the matrix optimization function is:
H:=∥gₓM_{XY}g,_{Y}^{t}∥_{F}²+λ_{X}Tr(g_{X}M_{XX}g_{X}^{t})+λ_{Y}Tr(g_{Y}M_{YY}g_{Y}^{t}),
and the optimization constraint is:
w_{xy(}M_{XY})=ν_{XY}
where M_{XY} is the drug-target interaction matrix and w_{xy} is a projection mapping operation and ν_{xy} is a two-dimensional vector, having a vector component for the drug-drug matrix and a vector component for the target-target matrix.

10. The method of claim 1, the method further comprising performing the optimization of the matrix optimization function until the interaction candidate entry is identified comprises:
minimizing a first distance between an entry in the drug-drug matrix to a drug in the drug-target interaction matrix, the first distance being measured according to the drug optimization function and/or minimizing a second distance between an entry in the target-target matrix to a target in the drug-target interaction matrix, the second distance being measured according to the target optimization function.

11. The method of claim 1, wherein the drug-drug matrix is a drug similarity matrix.

12. The method of claim 1, wherein the drug-drug matrix is a drug interaction matrix.

13. The method of claim 1, wherein the target-target matrix is a target similarity matrix.

14. The method of claim 1, wherein the target-target matrix is a target interaction matrix.

15. The method of claim 1, wherein at least one of the drug-drug matrix and the target-target matrix is an incomplete or sparse matrix.

16. The method of claim 1, wherein the matrix optimization function is scalable to be applied to a plurality of drug-drug matrices and to a plurality target-target matrices.

17. The method of claim 1, wherein identifying the sparse or incomplete entries in the drug-target interaction matrix comprises identifying entries having a [0, 1] value.

18. A computer-implemented method of performing completion of entries in a drug-target interaction matrix by predicting drug and target interactions from coupled datasets, the computer-implemented method comprising:
identifying, by a computer processor, incomplete entries in the drug-target interaction matrix;
accessing, by the computer processor, a drug-drug tensor and a target-target tensor;
determining, by the computer processor, a tensor optimization function for use in accessing the drug-drug tensor and for use in accessing the target-target tensor;
using the tensor optimization function, accessing, by the computer processor, a subset of entries in the drug-target interaction matrix and, using the tensor optimization function, accessing a plurality of slices of the drug-drug tensor and a plurality of slices of the target-target sensor;
performing, by the computer processor, an optimization of the matrix optimization function for each of the plurality of slices of the drug-drug tensor and for each of the plurality of slices of the target-target tensor, thereby producing an optimum candidate drug entry and optimum candidate target entry for each slice;
performing, by the computer processor, an optimization on the optimum candidate drug entries and on the optimum candidate target entries for each of the slices to identify a predicted interaction entry containing an optimum entry from the drug-drug tensor and an optimum entry from the target-target tensor, and populating one of the incomplete entries of the drug-target interaction matrix with the predicted interaction entry to form an updated drug-target interaction matrix; and
receiving, by the computer processor, subsequent drug and/or target data, comparing the subsequent drug and/or target interaction data to the updated drug-target interaction matrix and outputting one or more resulting interaction entries from the updated drug-target interaction matrix.

19. The computer-implemented method of claim 18, wherein the drug-drug tensor comprises a plurality of slices of drug-drug correlation data each differing in drug similarities, drug structure, drug functional characteristic, chemical environment response, drug interaction, or drug topology.

20. The computer-implemented method of claim 18, wherein the target-target tensor comprises a plurality of slices of target-target correlation data each differing in binary interaction, similarity score, or target type.

21. The computer-implemented method of claim 18, wherein the optimization expression is:
$$G := {‖ {g}_{X} {M}_{X Y} {g}_{Y}^{t} ‖}_{F}^{2} + {λ}_{X} {{\sum }^{{n}_{U}}}_{i = 1} {( {g}_{U} )}_{i , i} T r ( {g}_{X} {T}_{X X U}^{i} {g}_{X}^{t} ) + {λ}_{Y} {{\sum }^{{n}_{Z}}}_{j = 1} {( {g}_{Z} )}_{j , j} T r ( {g}_{Y} {T}_{Y Y Z}^{j} {g}_{Y}^{t} )$$
and an optimization constraint is:
w_{xy}(M_{XY})=ν_{XY}
where M_{XY} is the drug-target interaction matrix and w_{xy} is a projection mapping operation and ν_{xy} is a two-dimensional vector, having a vector component for the drug-drug matrix and a vector component for the target-target matrix.

22. The computer-implemented method of claim 18, wherein the tensor optimization function is a fixed function.

23. The computer-implemented method of claim 18, wherein the tensor optimization function is a Euclidean function or nuclear norm.

24. The computer-implemented method of claim 18, wherein the tensor optimization function is selected from the group consisting of a Mahalanobis distance, a Kempf-Ness function, a Morgan Fingerprint, an inverse of Jukes-Cantor distance, an Avalon fingerprint, an inverse of Mahalanobis distance, and a Kernel alignment ant.

25. A computer-implemented method predicting new interactions/relationships between a two sets of data based on known information, the method comprising:
obtaining a first tensor of a first data set, the first tensor comprising a plurality of different slices of the first data set, each slice containing a different relationship of the first data set;
obtaining a second tensor of a second data set, the second tensor comprising a plurality of slices of the second data set, each slice containing a different relationship of the second data set;
analyzing an interaction matrix containing interaction data between the first data set and the second data set to identify incomplete entries in the interaction matrix; and
performing an iterative minimization of an optimization function coupling the interaction matrix to the first tensor and to the second tensor to determine one or more predicted new interactions/relationships between the first data set and the second data set and updating the interaction matrix with the one or more predicted new interactions/relationships.

26. The computer-implemented method of claim 25, wherein the first data set comprises customer data and the second data set comprises financial services.

27. The computer-implemented method of claim 21, wherein the first data set comprises customer data and the second data set comprises on-streaming media content.

28. The computer-implemented method of claim 21, wherein the first data set comprises drug interaction data and second data set comprises drug target data.

29. The computer-implemented method of claim 21, wherein the first tensor and the second tensor are each 3D tensors.

30. The computer-implemented method of claim 21, wherein the first tensor and the second tensor each have dimensions great than 3D.
